(19) United States
(12) Patent Application Publication
(10) Pub. No.: US 2025/0215015 A1
(43) Pub. Date: Jul. 3, 2025

(54) COMPOSITIONS OF STAUROSPORINE ANALOGS AND USES THEREOF

(71) Applicant: THE REGENTS OF THE UNIVERSITY OF CALIFORNIA, Oakland, CA (US)

(72) Inventors: Milan Theodore MAKALE, La Jolla, CA (US); Wolfgang WRASIDLO, La Jolla, CA (US)

(21) Appl. No.: 19/076,514

(22) Filed: Mar. 11, 2025

Related U.S. Application Data
(63) Continuation of application No. PCT/US2023/074787, filed on Sep. 21, 2023.
(60) Provisional application No. 63/376,538, filed on Sep. 21, 2022.

(51) Int. Cl. C07D 498/22; A61K 9/107; A61K 31/553; A61P 35/00

(52) U.S. Cl. CPC C07D 498/22; A61K 9/1075; A61K 31/553; A61P 35/00

(57) ABSTRACT

Provided herein, inter alia, are compositions comprising a staurosporine analog and methods for their use in cancer treatment.

### RELATED APPLICATIONS

This application is a continuation of and claims priority to the International Patent Application No. PCT/US2023/074787, filed Sep. 21, 2023, which claims the benefit of U.S. Provisional Application No. 63/376,538 filed Sep. 21, 2022, the contents of each of which are incorporated herein in their entireties and for all purposes.

### BACKGROUND

Glioblastoma Multiforme (GBM) is the most common primary brain malignancy in adults, and high rates of mortality make this disease the third leading cause of cancer-related death among men 15-54 years of age and the fourth leading cause of cancer death for women 15-34 years of age. Multiple biological processes contribute to the aggressive nature of GBM; key among these are uncontrolled cellular proliferation, diffuse infiltration of brain parenchyma, and angiogenesis. The initial barrier to GBM treatment success and the focus of this proposal is resistance and local treatment failure after surgery, allowing tumor recurrence at the primary tumor site followed by expansion into adjacent normal brain. The major factors governing GBM resistance are intra-tumoral heterogeneity, activation of multiple signaling pathways, tumor cell drug efflux mechanisms, and perhaps most importantly, the inability of most compounds to attain therapeutic intra-tumoral concentrations even though GBMs are highly vascularized and have porous vessels. UCN-01, or 7-hydroxy staurosporine, can overcome chemoresistant tumors, is well-tolerated, has low toxicity and is approved for patient testing. UCN-01 is a potent staurosporine analog anti-tumor agent that is approved for patient testing, and is a superior radiochemotherapy sensitizer. UCN-01 even in non-cytotoxic doses markedly sensitizes tumor cells to radiation and chemotherapeutics. The potency of UCN-01 markedly exceeds that of standard agents and it engages a unique pattern of signaling pathways to drive apoptosis. However, UCN-01 is instantly bound by human alpha-1 acid glycoprotein (AAG) plasma protein with extremely high affinity and remains in circulation for remarkably extended periods of time. This greatly diminishes the availability of the free (active) compound so its in vivo effectiveness is compromised: when bound to AAG the UCN-01 molecule is not bioavailable and increasing the dose to sufficiently saturate AAG in the plasma elevates the risk of toxicity. The compositions and methods provided herein, inter alia, address these and other problems in the art.

### BRIEF SUMMARY

In one aspect, a compound, or a pharmaceutically acceptable salt thereof, is provided, having the formula:

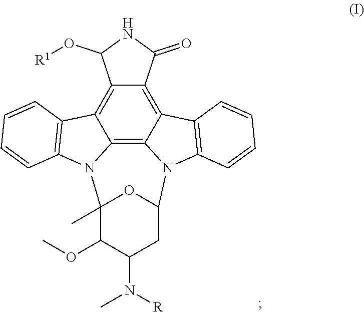

wherein
R and R¹ are independently hydrogen, halogen, —CX²₃, —CHX²₂, —CH₂X², —OCX²₃, —OCHX²₂, —OCH₂X², —CN, —SOₙ₂R², —SOᵥ₂NR²R³, —NR⁴NR²R³, —ONR²R³, —NR⁴C(O)NR²R³, —N(O)ₘ₂, —NR²R³, —C(O)R², —C(NH)R², —C(O)OR², —OC(O)R², —OC(O)OR², —C(O)NR²R³, —OC(O)NR²R³, —OR², —SR², —NR⁴SO₂R², —NR⁴C(O)R², —NR⁴C(O)OR²,
—NR²OR³, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R², R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH,
   —CONH₂, —OCCl₃, —OCF₃, —OCBr₃, —OCI₃, —OCHCl₂, —OCHBr₂, —OCHI₂, —OCH F₂, —OCH₂Cl,
   —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R³ substituents bonded to the same nitrogen atom may optionally be joined to form a substituted or unsubstituted heterocycloalkyl or substituted or unsubstituted heteroaryl;
   m2 and v2 are independently 1 or 2;
   n2 is an integer from 0 to 4; and
each X² is independently —Cl, —Br, —I, or —F;
wherein R and R¹ are not both hydrogen.

In some aspects, R is hydrogen,

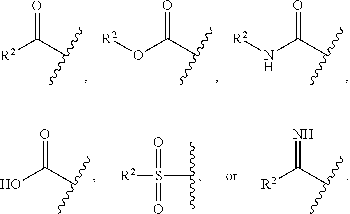

In some aspects, R² is hydrogen, substituted or unsubstituted C₁-C₄ alkyl, substituted or unsubstituted 3 to 6 membered heterocycloalkyl, unsubstituted phenyl, unsubstituted 5 to 6 membered heteroaryl, or
“\*MERGEFORMAT\*MERGEFORMAT —(CH₂CH₂O)ₙ-(unsubstituted C₁-C₄ alkyl), wherein n is an integer from 1 to 12.

In some aspects, R² is hydrogen, unsubstituted methyl, —CH₂CH₂OH, —CH₂-(unsubstituted phenyl), unsubstituted piperidinyl, substituted dioxanyl, unsubstituted phenyl, unsubstituted imidazolyl, unsubstituted oxazolyl, unsubstituted thiazolyl, unsubstituted pyrazinyl, —(CH₂CH₂O)₂—CH₃, —(CH₂CH₂O)₃—CH₃, or
“*\MERGEFORMAT\*MERGEFORMAT —(CH₂CH₂O)₄—CH₃.

In some aspects, R is hydrogen,

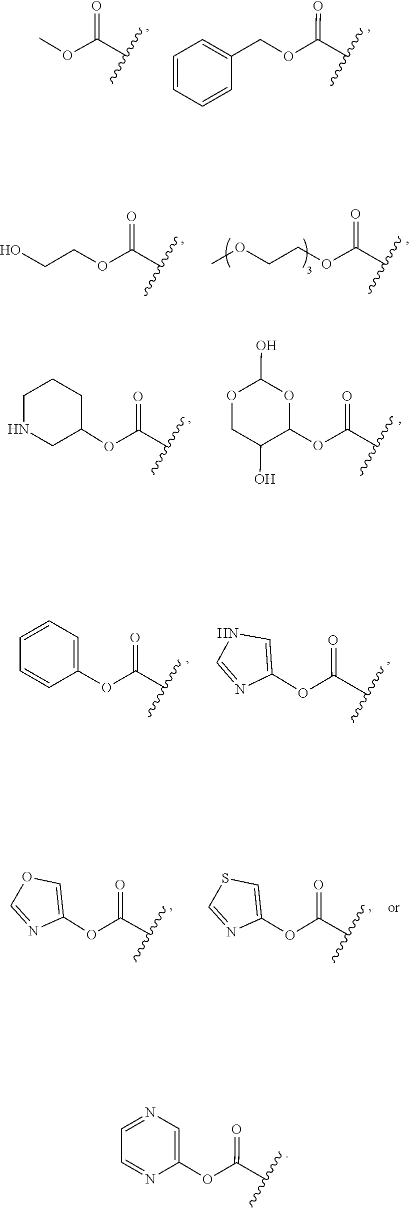

In some aspects, R¹ is hydrogen, unsubstituted C₁-C₄ alkyl, unsubstituted phenyl, or —(CH₂CH₂O)ₙ-(unsubstituted C₁-C₄ alkyl), wherein n is an integer from 1 to 12.

In some aspects, R¹ is hydrogen, unsubstituted methyl, unsubstituted butyl, unsubstituted phenyl, or —(CH₂CH₂O)₃—CH₃.

In some aspects, R or R¹ is a cleavable moiety.

In some aspects, R and R¹ are each a cleavable moiety.

In some aspects, the cleavable moiety is

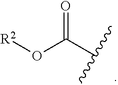

In some aspects, R² is unsubstituted C₁-C₄ alkyl, unsubstituted phenyl, or —(CH₂CH₂O)ₙ-(unsubstituted C₁-C₄ alkyl), wherein n is an integer from 1 to 12.

In some aspects, R² is unsubstituted methyl, unsubstituted butyl, unsubstituted phenyl, —(CH₂CH₂O)₂—CH₃, —(CH₂CH₂O)₃—CH₃,
“*\MERGEFORMAT\*MERGEFORMAT —(CH₂CH₂O)₄—CH₃, or —(CH₂CH₂O)₃—CH₂CH₃.

In some aspects, the compound having the formula:

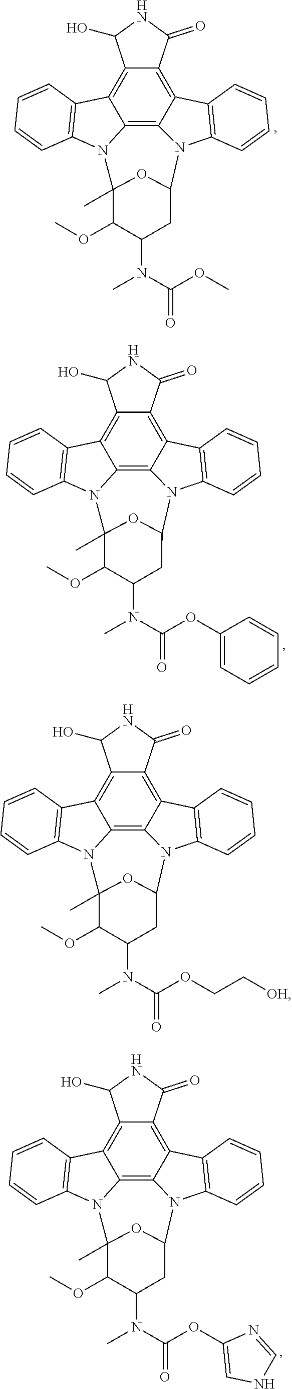 [SMILES: COC(=O)N(C)C1CC2OC(C)(C1OC)n1c3ccccc3c3c4c(c5c6ccccc6n2c5c31)C(=O)NC4O ; COC1C(N(C)C(=O)Oc2ccccc2)CC2OC1(C)n1c3ccccc3c3c4c(c5c6ccccc6n2c5c31)C(=O)NC4O ; COC1C(N(C)C(=O)OCCO)CC2OC1(C)n1c3ccccc3c3c4c(c5c6ccccc6n2c5c31)C(=O)NC4O ; COC1C(N(C)C(=O)Oc2c[nH]cn2)CC2OC1(C)n1c3ccccc3c3c4c(c5c6ccccc6n2c5c31)C(=O)NC4O]

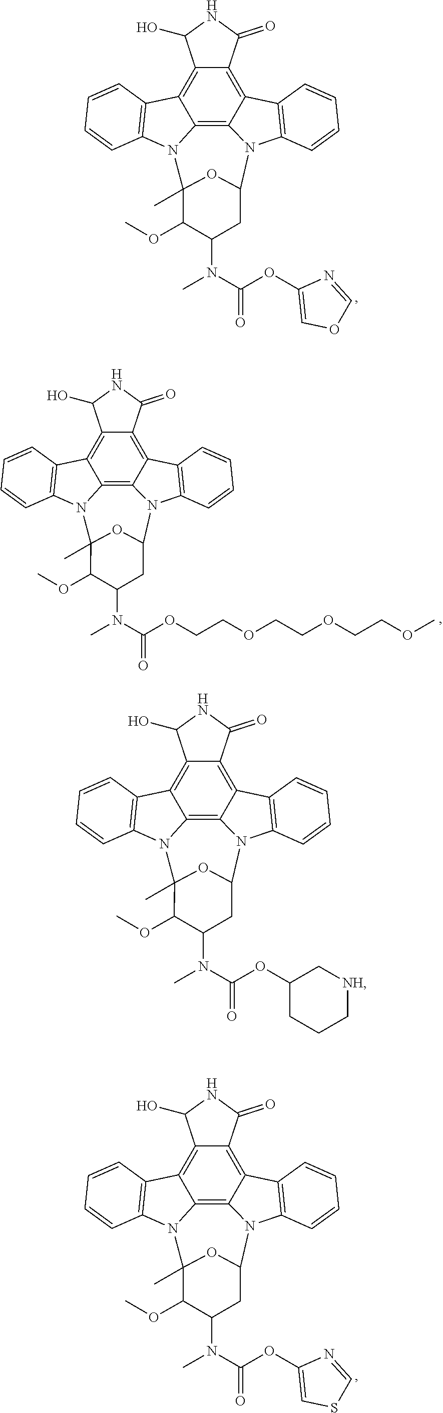 [SMILES: COC1C(N(C)C(=O)Oc2cocn2)CC2OC1(C)n1c3ccccc3c3c4c(c5c6ccccc6n2c5c31)C(=O)NC4O ; COCCOCCOCCOC(=O)N(C)C1CC2OC(C)(C1OC)n1c3ccccc3c3c4c(c5c6ccccc6n2c5c31)C(=O)NC4O ; COC1C(N(C)C(=O)OC2CCCNC2)CC2OC1(C)n1c3ccccc3c3c4c(c5c6ccccc6n2c5c31)C(=O)NC4O ; COC1C(N(C)C(=O)Oc2cscn2)CC2OC1(C)n1c3ccccc3c3c4c(c5c6ccccc6n2c5c31)C(=O)NC4O]

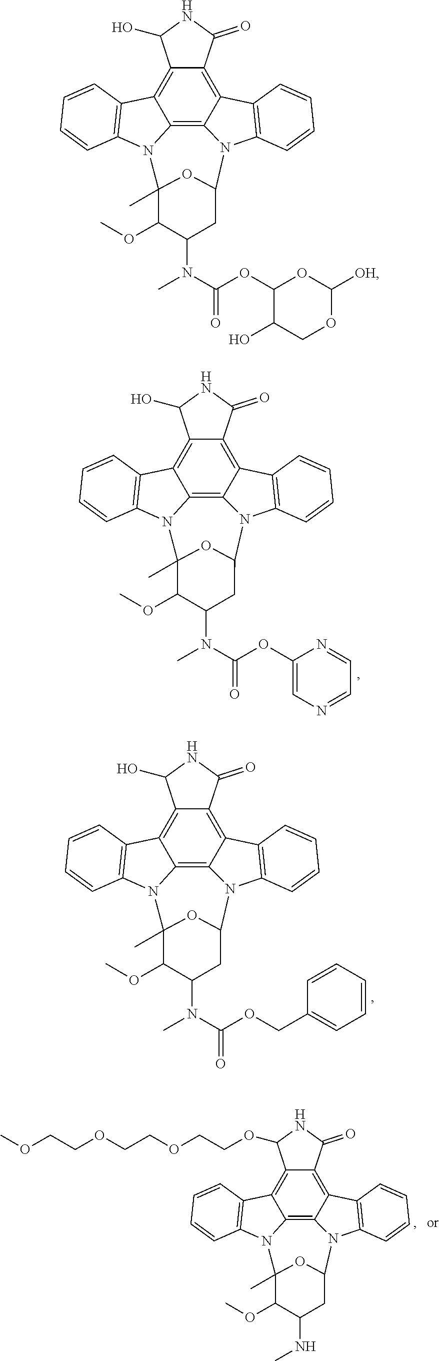 [SMILES: COC1C(N(C)C(=O)OC2OC(O)OCC2O)CC2OC1(C)n1c3ccccc3c3c4c(c5c6ccccc6n2c5c31)C(=O)NC4O ; COC1C(N(C)C(=O)Oc2cnccn2)CC2OC1(C)n1c3ccccc3c3c4c(c5c6ccccc6n2c5c31)C(=O)NC4O ; COC1C(N(C)C(=O)OCc2ccccc2)CC2OC1(C)n1c3ccccc3c3c4c(c5c6ccccc6n2c5c31)C(=O)NC4O ; CNC1CC2OC(C)(C1OC)n1c3ccccc3c3c4c(c5c6ccccc6n2c5c31)C(=O)NC4OCCOCCOCCOC]

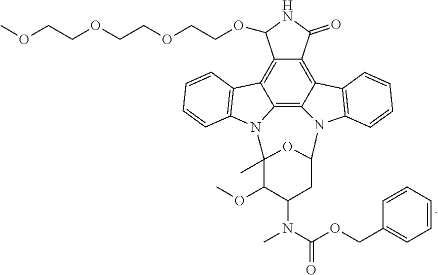 [SMILES: COCCOCCOCCOC1NC(=O)c2c1c1c3ccccc3n3c1c1c2c2ccccc2n1C1CC(N(C)C(=O)OCc2ccccc2)C(OC)C3(C)O1]

In some aspects, a pharmaceutical composition is provided comprising the compound of the disclosure and a pharmaceutically acceptable excipient. In some aspects, the compound is comprised in a stable micelle. In some aspects, the micelle comprises a polyethylene glycol (PEG) moiety. In some aspects, a method for treating a subject suffering from cancer or a tumor is provided, the method comprising administering a therapeutically effective amount of the pharmaceutical composition of the disclosure to the subject.

In some aspects, the method further comprises administering to the subject a chemotherapy or a radiotherapy. In some aspects, the cancer is a head and neck cancer (HNSCC), breast cancer, or brain cancer. In some aspects, the brain cancer GBM.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows that UCN-01 radiosensitizes at mildly cytotoxic doses. Bar graph of radiation dose and UCN-01 dose (nM) vs percent viable U87 GBM cells. Red columns for 0 Gy and purple for 5 Gy—a clinically relevant dose.

FIG. 2 shows that UCN-01 potentiated temozolomide (TMZ) in U87 glioblastoma cells.

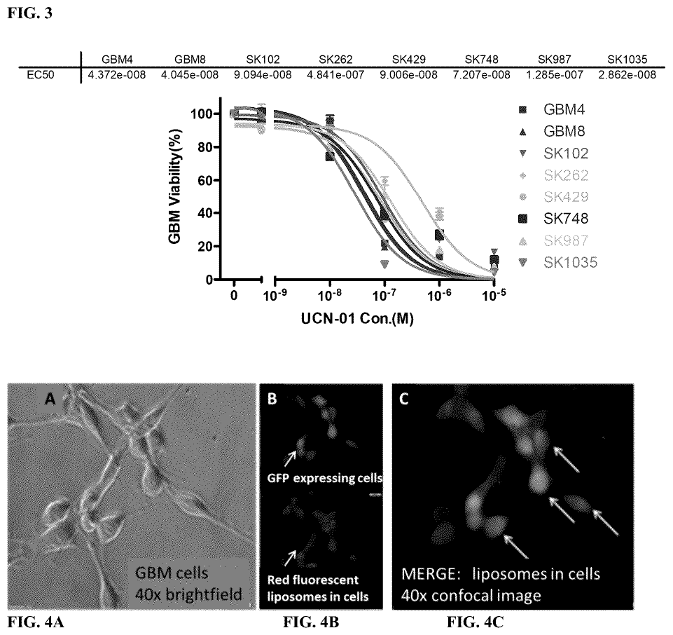

FIG. 3 shows that UCN-01 alone was active against a range of tumorsphere derived glioblastoma lines.

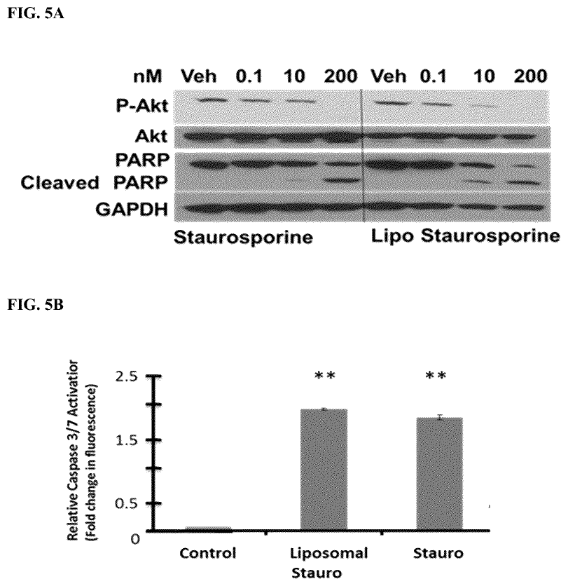

FIGS. 4A-4C show that liposomes entered GBM cells in vitro. FIG. 4A: brightfield image of cells incubated with empty fluorescent liposomes. FIG. 4B: confocal images green channel and red channel (lower). FIG. 4C: merge of green and red; arrows point to cells with labeled liposomes.

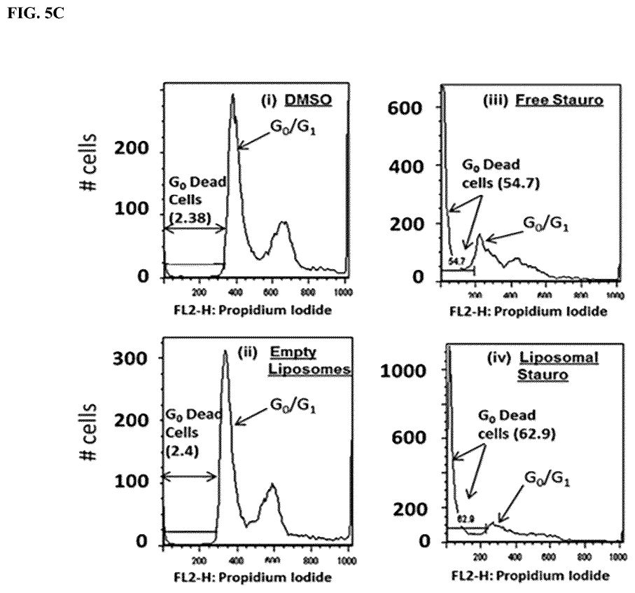

FIGS. 5A-5C show that in a mechanistic context free Staurosporine (STS) and Lipostaurosporine (LSTS) both induce apoptosis in GBM cells and inhibits phosphorylation of Akt. FIG. 5A: Western blot analysis for Akt, P-Akt, cleaved PARP. U87 cells incubated with 200 nM, 10 nM and 0.1 nM of free or liposomal STS for 32 hrs. Representative blot from two independent experiments is shown. FIG. 5B: Caspase3/7 activity in U87 cells incubated for 12 hours with DMSO control, 200 nM free STS, and LSTS. Empty liposomes had no fluorescence. FIG. 5C: FACS analysis for DNA showing G0 shift. (p<0.05).

FIGS. 6A-6B show that liposomes loaded with STS preferentially localized in tumor tissue. FIG. 6A: Liposomes (red) tumor microvessels. 20× confocal. FIG. 6B: Tissue specific liposomal fluorescence averaged and normalized for two mice. Bars depict range.

FIGS. 7A-7B show that LSTS inhibited the growth of established (40-50 mm³) U87 flank tumors with no overt toxicity over 3 weeks. FIG. 7A: PBS control (♦), empty liposomes (▪), or LSTS (▴, 0.8 mg/kg) injected i.v 3× a week over 3 weeks. The x-axis is days after tumor implantation. n=5, p=0.00043. FIG. 7B: Images of nu/nu mice implanted SC with U87 cells.

FIG. 8 shows that LSTS had no evident toxicity according to body weight and liver enzymes. Top, bar graph shows weight change in mice bearing U87 GBM flank tumors, after treatment with PBS, LSTS, and free STS. Weight declined with STS but not PBS or LSTS. Bottom panel is bar graph depicting liver enzymes ALT/AST before and after treatment. For free STS the post treatment levels were flagged as abnormal (stars). ALT/AST enzyme levels were normal with empty liposomes (control) and LSTS. Reference levels shown by dashed lines.

FIG. 9 shows STS liposomes inside primary GBM tumor. Liposomes: - indicated by white arrows. Microvessels.

FIGS. 10A-10B shows that LSTS significantly extended the survival of mice with orthotopic GBM. FIG. 10A shows the survival effect of the current front line GBM therapeutic TMZ in C57BL6 mice bearing orthotopic GL262 GBM tumors. Line 2 is survival of treated mice, line 1 is control, and by 42 days all animals expired. (Adapted from Bai et al, 2011, Neuro-Oncology.) FIG. 10B shows the survival effect of avB3 LSTS in 10 nu/nu mice with orthotopic GBM8 patient derived GBM tumors: line 2, controls: line 1.

FIG. 11 shows human GBM heavily expressed avB3. Left panel shows staining for avB3 in human GBM, while staining in middle panel is for normal cortex, and right panel is normal white matter. Bars=100 μm. This may have implications for avB3 targeting of liposomes.

FIGS. 12A-12C show pegylation decreases UCN-01 cytotoxicity in MDA-MB-231 cells (FIG. 12A), HT-3 cells (FIG. 12B), and HeLa cells (FIG. 12C).

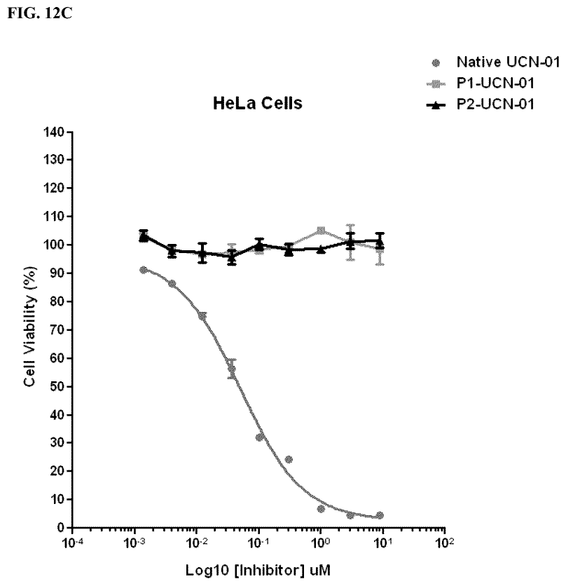

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to
“*\MERGEFORMAT\*MERGEFORMAT —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃,
“*\MERGEFORMAT\*MERGEFORMAT —CH₂—CH₂—N(CH₃)—CH₃, —CH₂—S—CH₂—CH₃, —CH₂—S—CH₂, —S(O)—CH₃, —CH₂—CH₂—S(O)₂—CH₃,
“*\MERGEFORMAT\*MERGEFORMAT —CH═CHO—CH₃, —Si(CH₃)₃, —CH₂—CH═N—OCH₃, —CH═CH—N(CH₃)—CH₃, —O—CH₃,
“*\MERGEFORMAT\*MERGEFORMAT —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula
“*\MERGEFORMAT\*MERGEFORMAT —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as
“*\MERGEFORMAT\*MERGEFORMAT —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

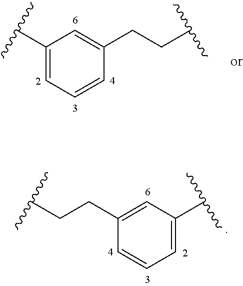 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃,
“*\MERGEFORMAT\*MERGEFORMAT —CF₃, —CCl₃, —CBr₃, —CI₃, —CN, —CHO, —OH, —NH₂, —COOH, —CONH₂, —NO₂, —SH, —SO₂CH₃
“\*MERGEFORMAT\*MERGEFORMAT —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, -halogen,
“*\MERGEFORMAT\*MERGEFORMAT —SiR′R″R′″, —OC(O)R′, —C(O)R′, —CO₂R′, —CONR′R″, —OC(O)NR′R″, —NR″C(O)R′,
“*\MERGEFORMAT\*MERGEFORMAT —NR′—C(O)NR″R′″, —NR″C(O)₂R′, —NR—C(NR′R″R′″)=NR″″, —NR—C(NR′R″)═NR′″″, —S(O)R′,
“*\MERGEFORMAT\*MERGEFORMAT —S(O)₂R′, —S(O)₂NR′R″, —NRSO₂R′, —NR′NR″R″, —ONR′R″, —NR′C(O)NR″NR″′R″″, —CN,
“*\MERGEFORMAT\*MERGEFORMAT —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g.,
“*\MERGEFORMAT\*MERGEFORMAT —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′,
“*\MERGEFORMAT\*MERGEFORMAT -halogen, —SiR′R″R′″, —OC(O)R′, —C(O)R′, —CO₂R′, —CONR′R″, —OC(O)NR′R″, —NR″C(O)R′,
“*\MERGEFORMAT\*MERGEFORMAT —NR′—C(O)NR″R′″, —NR″C(O)₂R′, —NR—C(NR′R″R′″)=NR″″, —NR—C(NR′R″)═NR′″, —S(O)R′,
“*\MERGEFORMAT\*MERGEFORMAT —S(O)₂R′, —S(O)₂NR′R″, —NRSO₂R′, —NR′NR″R′″, —ONR′R″, —NR′C(O)NR″NR″′R″″, —CN,
“*\MERGEFORMAT\*MERGEFORMAT —NO₂, —R′, —N₃, —CH(Ph)₂, fluoro(C₁-C₄)alkoxy, and fluoro(C₁-C₄)alkyl, —NR′SO₂R″,
“*\MERGEFORMAT\*MERGEFORMAT —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula
“*\MERGEFORMAT\*MERGEFORMAT —(CRR′)ₛ—X′—(C″R″R′″)_{d}—, where s and d are independently integers of from 0 to 3, and X′ is
“*\MERGEFORMAT\*MERGEFORMAT —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{1A.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW1} or R^{LWW.1}, respectively. In turn, each first substituent group (e.g., R^{1.1}, R^{2.1}, R^{3.1}, R^{4.1}, R^{5.1} . . . R^{100.1}. R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R″ group is phenyl substituted by R^{WW.1}, which is methyl, the methyl group may be further substituted to form groups including but not limited to:

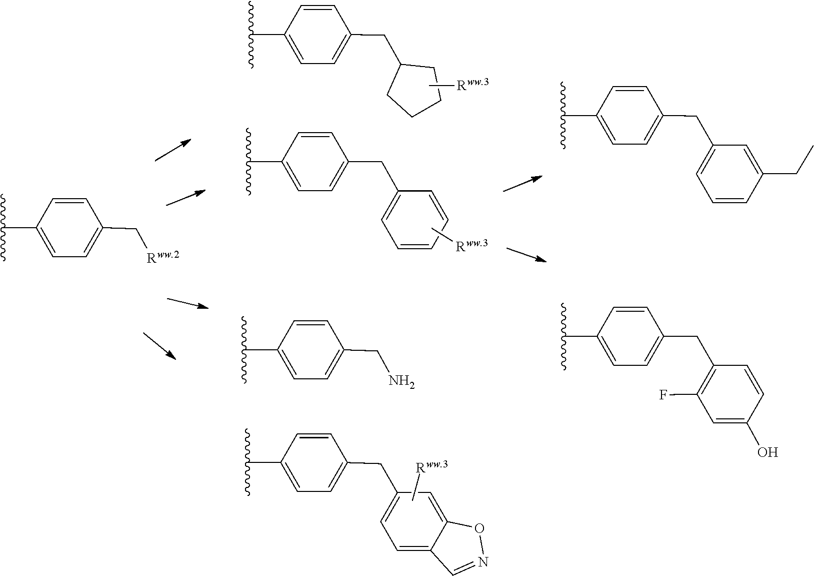

R^{WW.1} is independently oxo, halogen, —CX^{WW.1}₃, —CHX^{WW.1}₂, —CH₂X^{WW.1},
“*\MERGEFORMAT\* MERGEFORMAT —OCX^{WW.1}₃, —OCH₂X^{WW.1}, —OCHX^{WW.1}₂, —CN, —OH, —NH₂, —COOH, —CO NH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂,
“*\MERGEFORMAT\* MERGEFORMAT —CH₂X^{WW.1}, —OCX^{WW.1}₃, —OCH₂X^{WW.1}, —OCHX^{WW.1}₂, —CN, —OH, —NH₂, —COOH, —CONH₂,
“*\MERGEFORMAT\* MERGEFORMAT —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.
R^{WW.2} is independently oxo, halogen, —CX^{WW.2}₃, —CHX^{WW.2}₂, —CH₂X^{WW.2},
“*\MERGEFORMAT\* MERGEFORMAT —OC^{WW.2}₃, —OCH₂X^{WW.2}, —OCHX^{WW.2}₂, —CN, —OH, —NH₂, —COOH, —CO NH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂,
“*\MERGEFORMAT\* MERGEFORMAT —CH₂X^{WW.2}, —OCX^{WW.2}₃, —OCH₂X^{WW.2}, —OCHX^{WW.2}₂, —CN, —OH, —NH₂, —COOH, —CONH₂,
“*\MERGEFORMAT\* MERGEFORMAT —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3},
“*\MERGEFORMAT\* MERGEFORMAT —OCX^{WW.3}, —OCH₂X^{WW.3}, —OCHX^{WW.3}₂, —CN, —OH, —NH₂, —COOH, —CO NH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R″ substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃ CHX^{LWW.1}₂, —CH₂X^{LWW.1},
“*\MERGEFORMAT\* MERGEFORMAT —OCX^{LWW1}₃, —OCH₂X^{LWW.1}, —OCHX^{LWW.1}₂, —CN, —OH, —NH₂, —COOH, —CONH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃,
“*\MERGEFORMAT\* MERGEFORMAT —CHX^{LWW.1}₂, —CH₂X^{LWW.1}, —OCX^{LWW.1}₃, OCH₂X^{LWW.1}, —OCHX^{LWW.1}₂—CN, —OH, —NH₂,
“*\MERGEFORMAT\* MERGEFORMAT —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH,
“*\MERGEFORMAT\*MERGEFORMAT —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2},
“*\MERGEFORMAT\* MERGEFORMAT —OCX^{LWW.2}₃, —OCH₂X^{LWW.2}, OCHX^{LWW.2}₂, —CN, —OH, —NH₂, —COOH, —CONH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃
“*\MERGEFORMAT\* MERGEFORMAT —CHX^{LWW.2}₂, —CH₂X^{LWW.2}, OCX^{LWW.2}₃, —OCH₂X^{LWW.2}, OCHX^{LWW.2}₂, —CN, —OH, —NH₂,
“*\MERGEFORMAT\* MERGEFORMAT —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH,
“*\MERGEFORMAT\*MERGEFORMAT —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.
R^{LWW.3} is independently oxo, halogen, —CX^{LWW.3}₃, —CHX^{LWW.3}₂, —CH₂X^{LWW.3},
“*\MERGEFORMAT\* MERGEFORMAT —OCX^{LWW.3}₃, —OCH₂X^{LWW.3}, OCHX^{LWW.3}₂, —CN, —OH, —NH₂, —COOH, —CONH₂, —NO₂, —SH,
“*\MERGEFORMAT\*MERGEFORMAT —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂,
“*\MERGEFORMAT\* MERGEFORMAT —CH₂X^{WW}, —OCX^{WW}₃, —OCH₂X^{WW}, —OCHX^{WW}₂, —CN, —OH, —NH₂, —COO H, —CONH₂, —NO₂,
“*\MERGEFORMAT\*MERGEFORMAT —SH, —SO₃H, —OSO₃H, —SO₂NH₂, —NHNH₂, —ONH₂, —NHC(O)NHNH₂, —NHC(O)NH₂,
“*\MERGEFORMAT\*MERGEFORMAT —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—,
“*\MERGEFORMAT\*MERGEFORMAT —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g., phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g., a first linker or second linker), or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

The term “capable of binding” as used herein refers to a moiety (e.g., a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

The term “non-nucleophilic base” as used herein refers to any sterically hindered base that is a poor nucleophile.

The term “nucleophile” as used herein refers to a chemical species that donates an electron pair to an electrophile to form a chemical bond in relation to a reaction. All molecules or ions with a free pair of electrons or at least one pi bond can act as nucleophiles.

The term “strong acid” as used herein refers to an acid that is completely dissociated or ionized in an aqueous solution. Examples of common strong acids include hydrochloric acid (HCl), nitric acid (HNO₃), sulfuric acid (H₂SO₄), hydrobromic acid (HBr), hydroiodic acid (HI), perchloric acid (HCLO₄), or chloric acid (HClO₃).

The term “carbocation stabilizing solvent” as used herein refers to any polar protic solvent capable of forming dipole-dipole interactions with a carbocation, thereby stabilizing the carbocation.

A “nanoparticle,” as used herein, is a particle wherein the longest diameter is less than or equal to 1000 nanometers. The longest dimension of the nanoparticle may be referred to herein as the length of the nanoparticle. The shortest dimension of the nanoparticle may be referred to herein refer as the width of the nanoparticle. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. In embodiments, the nanoparticle has the shape of a sphere, rod, cube, triangular, hexagonal, cylinder, spherocylinder, or ellipsoid.

An “inorganic nanoparticle” refers to a nanoparticle without carbon. For example, an inorganic nanoparticle may refer to a metal or metal oxide thereof (e.g., gold nanoparticle, iron nanoparticle) silicon and oxides thereof (e.g., a silica nanoparticle), or titanium and oxides thereof (e.g., titanium dioxide nanoparticle). In embodiments, the inorganic nanoparticle is a silica nanoparticle. The inorganic nanoparticle may be a metal nanoparticle. When the nanoparticle is a metal, the metal may be titanium, zirconium, gold, silver, platinum, cerium, arsenic, iron, aluminum or silicon. The metal nanoparticle may be titanium, zirconium, gold, silver, or platinum and appropriate metal oxides thereof. In embodiments, the nanoparticle is titanium oxide, zirconium oxide, cerium oxide, arsenic oxide, iron oxide, aluminum oxide, or silicon oxide. The metal oxide nanoparticle may be titanium oxide or zirconium oxide. The nanoparticle may be titanium. The nanoparticle may be gold. In embodiments, the metal nanoparticle is a gold nanoparticle. In embodiments, the inorganic nanoparticle may further include a moiety which contains carbon.

The term “silica” is used according to its plain and ordinary meaning and refers to a composition (e.g., a solid composition such as a crystal, nanoparticle, or nanocrystal) containing oxides of silicon such as Si atoms (e.g., in a tetrahedral coordination) with 4 oxygen atoms surrounding a central Si atom. Nanoparticles may be composed of at least two distinct materials, one material (e.g., insoluble drug) forms the core and the other material forms the shell (e.g., silica) surrounding the core; when the shell includes Si atoms, the nanoparticle may be referred to as a silica nanoparticle. A silica nanoparticle may refer to a particle including a matrix of silicon-oxygen bonds wherein the longest diameter is typically less than or equal to 1000 nanometers.

A functionalized silica nanoparticle, as used herein, may refer to the post hoc conjugation (i.e. conjugation after the formation of the silica nanoparticle) of a moiety to the hydroxyl surface of a nanoparticle. For example, a silica nanoparticle may be further functionalized to include additional atoms (e.g., nitrogen) or chemical entities (e.g., polymeric moieties or bioconjugate group). For example, when the silica nanoparticle is further functionalized with a nitrogen containing compound, one of the surface oxygen atoms surrounding the Si atom may be replaced with a nitrogen containing moiety.

The term “polymeric” refers to a molecule including repeating subunits (e.g., polymerized monomers). For example, polymeric molecules may be based upon polyethylene glycol (PEG), poly[amino(1-oxo-1,6-hexanediyl)], poly(oxy-1,2-ethanediyloxycarbonyl-1,4-phenylenecarbonyl), tetraethylene glycol (TEG), polyvinylpyrrolidone (PVP), poly(xylene), or poly(p-xylylene). See, for example, “Chemistry of Protein Conjugation and Cross-Linking” Shan S. Wong CRC Press, Boca Raton, Fla., USA, 1993; “BioConjugate Techniques” Greg T. Hermanson Academic Press, San Diego, Calif, USA, 1996; “Catalog of Polyethylene Glycol and Derivatives for Advanced PEGylation, 2004” Nektar Therapeutics Inc, Huntsville, Ala., USA, which are incorporated by reference in their entirety for all purposes.

The term “poloxamer” is used in accordance with its meaning in the art of polymer chemistry and refers to a triblock copolymer composed of a central hydrophobic block (e.g., polyoxypropylene) flanked by two hydrophilic blocks (e.g., polyoxyethylene). Poloxamers may be customized by adjusting the degree of hydrophobicity and/or hydrophilicity by extending or retracting the length of the blocks. Non-limiting examples of poloxamers include poloxomer 407, poloxomer 188, poloxomer 127, or poloxomer 388. Certain poloxomers are understood to be safe for use in humans, see for example Singh-Joy and McLain (Int J Toxicol. 2008; 27 Suppl 2:93-128) which is incorporated by reference in its entirety for all purposes.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “branched polymer” is used in accordance with its meaning in the art of polymer chemistry and refers to a molecule including repeating subunits, wherein at least one repeating subunit (e.g., polymerizable monomer) is covalently bound to an additional subunit substituent (e.g., resulting from a reaction with a polymerizable monomer). For example a branched polymer has the formula:

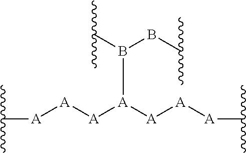

wherein ‘A’ is the first repeating subunit and ‘B’ is the second repeating subunit. In embodiments, the first repeating subunit (e.g., polyethylene glycol) is optionally different than the second repeating subunit (e.g., polymethylene glycol).

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g., -A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together.

The term “amphiphilic polymer” as used herein refers to a polymer containing both hydrophilic and hydrophobic portions. In embodiments, the hydrophilic to hydrophobic portions are present in a 1 to 1 mass ratio. In embodiments, the hydrophilic to hydrophobic portions are present in a 1 to 2 mass ratio. In embodiments, the hydrophilic to hydrophobic portions are present in a 1 to 5 mass ratio. In embodiments, the hydrophilic to hydrophobic portions are present in a 2 to 1 mass ratio. In embodiments, the hydrophilic to hydrophobic portions are present in a 5 to 1 mass ratio. An amphiphilic polymer may be a diblock or triblock copolymer. In embodiments, the amphiphilic polymer may include two hydrophilic portions (e.g., blocks) and one hydrophobic portion (e.g., block). In embodiments, the hydrophilic block to hydrophobic to hydrophilic ratio is 1 to 1 to 1. In embodiments, the hydrophilic block to hydrophobic to hydrophilic ratio is 1.8 to 1 to 1.8. In embodiments, the hydrophilic block to hydrophobic to hydrophilic ratio is 2 to 1 to 2. In embodiments, the hydrophilic block to hydrophobic to hydrophilic ratio is 1 to 1 to 2.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

The term “sonicating” as used herein refers to the process of applying sound energy to agitate particles in a sample. In situ, sonicating is applied using a sonicator such as an ultrasonic bath or an ultrasonic probe. In embodiments, the sound energy is at least 20 kHz. In embodiments, the sound energy is greater than 20 kHz. In embodiments, the sound energy is about 20 to about 40 kHz.

The term “macrolide” is used in accordance with its ordinary meaning in chemistry, and refers to a macrocyclic lactone ring to which one or more deoxy sugars is attached. Non-limiting examples of macrolides include erythromycin, clarithromycin, fidaxomicin, or azithromycin.

The term “steroid” is used in accordance with its plain ordinary meaning and refers to a a class of tetracyclic compounds with, three cyclohexane and one cyclopentane ring arranged with the structural formula:

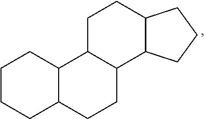 [SMILES: C1CCC2C(C1)CCC1C3CCCC3CCC21]

which is optionally substituted and may include one or more points of non-saturation (i.e. double bonds) within one or more of the rings. Steroids can vary in the number of functional groups or methyl groups attached to the rings, or differ in the level of saturation within the rings. Additional non limiting examples of steroids include cholesterol, cholic acid, progesterone, testosterone, or estradiol.

The phrase “average molecular weight” refers to the weight average molecular weight of a polymer as determined by gel permeation chromatography (also known as GPC or size exclusion chromatography (SEC)) using tetrahydrofuran (THF) as the solvent and using a molecular weight calibration curve using polystyrene standards.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. In embodiments, the nonspecific nucleic acid sequence does not encode a biological function. In embodiments, the nonspecific nucleic acid sequence is a scrambled nucleic acid sequence. A “scrambled nucleic acid sequence” as provided herein is a recombinant nucleic acid sequence that includes nucleotides randomly linked to each other in vitro. Scrambled nucleic acid sequences are commonly used in the art as control or reference sequences relative to the activity (biological function) of test nucleic acid sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natd. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region; also referred to herein as “Fc domain”) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. In embodiments, the Fc region includes a constant heavy chain domain 3 (CH3 domain) and a constant heavy chain domain 2 (CH2 domain).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “biparatopic antibody” provided herein is used according to its common meaning in the biological arts, and refers to a bispecific antibody including two antigen binding domains, each of which recognizes unique, non-overlapping epitopes on the same target antigen. Compared to monoclonal antibodies, biparatopic antibodies may exhibit a superior ability to promote receptor clustering, which may in turn result in improved receptor internalization, lysosomal trafficking, and receptor down regulation and therefore improved drug potency. In embodiments, a biparatopic antibody includes a first antigen binding domain including a heavy chain and a light chain; and a second antigen binding domain including a heavy chain and a light chain. In embodiments, the heavy chain of the first antigen binding domain and the heavy chain of the second antigen binding domain are different. In embodiments, the light chain of the first antigen binding domain and the light chain of the second antigen binding domain are different. In embodiments, a biparatopic antibody includes a first heavy chain, a first light chain, a second heavy chain and a second light chain, wherein the first heavy chain and the second heavy chain are different and wherein the first light chain and the second light chain are different.

The term “antigen” as provided herein refers to molecules capable of binding to the antigen binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen. The antibodies provided herein may be biparatopic antibodies. In embodiments, the biparatopic antibody includes a first paratope binding a first epitope and a second paratope binding a second epitope. In embodiments, the first paratope and the second paratope are different. In further embodiments, the first epitope and the second epitope are different.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain (e.g., a VH or a VL domain). Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

An “affibody” as described herein is commonly well known in the art and refers to small, robust proteins engineered to bind to a large number of target proteins or peptides with high affinity, by imitating monoclonal antibodies. Affibodies are therefore a member of the family of antibody mimetics. In embodiments, an affibody is a molecule including of three alpha helices with about 58 amino acids and a molar mass of about 6 kDa.

A “single domain antibody” as provided herein refers to an antibody fragment including a single monomeric variable antibody domain (e.g., a VH or a VL domain). Like a whole antibody, a single domain antibody is able to bind selectively to a specific antigen. The molecular weight of a single domain antibody is 12-15 kDa, single domain antibody. In embodiments, a single domain antibody is a variable heavy chain domain. In embodiments, a single domain antibody includes a variable heavy chain domain. In embodiments, a single domain antibody is a variable light chain domain. In embodiments, a single domain antibody includes a variable light chain domain. Non-limiting examples of single domain antibodies include camelid-derived VHH fragments and VNAR (variable immunoglobulin new antigen receptor) fragments. In embodiments, the single-domain antibody is a peptide domain of about 110 amino acids.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a ligand binding domain (e.g., receptor or antibody, antibody variant, antibody region or fragment thereof).

“Contacting” is used in accordance with its plain ordinary meaning and refers to the process of allowing at least two distinct species (e.g., chemical compounds including biomolecules or cells, an antibody provided herein and its epitope) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

As used herein, the term “lipid” is used in accordance with its plain ordinary meaning and refers to a micro biomolecule that is soluble in non-polar solvents. Non-polar solvents are typically hydrocarbons used to dissolve other naturally occurring hydrocarbon lipid molecules that do not (or do not easily) dissolve in water, including fatty acids, waxes, sterols, fat-soluble vitamins (such as vitamins A, D, E, and K), monoglycerides, diglycerides, triglycerides, and phospholipids. The functions of lipids include storing energy, signaling, and acting as structural components of cell membranes. Lipids have applications in the cosmetic and food industries as well as in nanotechnology.

As used herein, the term “phospholipid” is used in accordance with its plain ordinary meaning and refers to a class of lipids whose molecule has a hydrophilic “head” containing a phosphate group, and two hydrophobic “tails” derived from fatty acids, joined by a glycerol molecule. Marine phospholipids typically have omega-3 fatty acids EPA and DHA integrated as part of the phospholipid molecule. The phosphate group may be modified with simple organic molecules such as choline, ethanolamine or serine. Phospholipids are a key component of all cell membranes. They may form lipid bilayers because of their amphiphilic characteristic. In eukaryotes, cell membranes also contain another class of lipid, sterol, interspersed among the phospholipids. The combination provides fluidity in two dimensions combined with mechanical strength against rupture. Purified phospholipids are produced commercially and have found applications in nanotechnology and materials science.

As used herein, the term “lipid bilayer” or “phospholipid bilayer” is used in accordance with its plain ordinary meaning and refers to a polar membrane made of two layers of lipid molecules. These lipid bilayers are flat sheets that can form a continuous barrier around cells. Phospholipid bilayers are composed of amphiphilic phospholipids that have a hydrophilic phosphate head group and a hydrophobic tail consisting of two fatty acid chains. The phosphate head group of a phospholipid can alter the surface chemistry of the bilayer. In addition, the fatty acid tails can affect membrane properties (e.g., phase of the bilayer).

As used herein, the term “liposome” is used in accordance with its plain ordinary meaning and refers to a spherical vesicle having at least one lipid bilayer. The liposome can be used as a drug delivery vehicle for administration of nutrients and pharmaceutical drugs, such as lipid nanoparticles in mRNA vaccines, and DNA vaccines. Liposomes can be prepared by disrupting biological membranes (such as by sonication). Liposomes are most often composed of phospholipids, especially phosphatidylcholine, but may also include other lipids, such as egg phosphatidylethanolamine, so long as they are compatible with lipid bilayer structure. A liposome design may employ surface ligands for attaching to unhealthy tissue. The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. A multivesicular liposome is a vesicle that contains one or more smaller vesicles. Liposomes should not be confused with lysosomes, or with micelles and reverse micelles composed of monolayers.

As used herein, the term “vesicles” or “lipid vesicles” is used in accordance with its plain ordinary meaning and refers to a structure within or outside a cell, consisting of liquid or cytoplasm enclosed by a lipid bilayer. The vesicles form naturally during the processes of secretion (exocytosis), uptake (endocytosis) and transport of materials within the plasma membrane. Alternatively, they may be prepared artificially, in which case they are called liposomes (not to be confused with lysosomes). If there is only one phospholipid bilayer, they are called unilamellar liposome vesicles; otherwise they are called multilamellar. The membrane enclosing the vesicle is also a lamellar phase, similar to that of the plasma membrane, and intracellular vesicles may fuse with the plasma membrane to release their contents outside the cell. The vesicles may also fuse with other organelles within the cell. A vesicle released from the cell is known as an extracellular vesicle. The vesicles perform a variety of functions. Because it is separated from the cytosol, the inside of the vesicle may be made to be different from the cytosolic environment. For this reason, the vesicles are a basic tool used by the cell for organizing cellular substances. The vesicles are involved in metabolism, transport, buoyancy control, and temporary storage of food and enzymes. The vesicles may also act as chemical reaction chambers.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “plasmid,” “expression vector,” or “viral vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. Suitable viral vectors contemplated herein include, for example, lentiviral vectors and onco-retroviral vectors.

As used herein, the term “expression” is used in accordance with its plain ordinary meaning and refers to a step involved in the production of the polypeptide including, but not limited to, transcription, post-transcriptional modification, translation, post-translational modification, and secretion. Expression may be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient”, “patient in need thereof”, “subject”, or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

The terms “disease” or “condition” refer to a state of being or health status of a patient or subject capable of being treated with a compound, pharmaceutical composition, or method provided herein. In embodiments, the disease is cancer (e.g., lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the term “solid cancer” refers to a solid mass of cancer cells that grow in organ systems and can occur anywhere in the body, Solid cancers include, but are not limited to bile duct cancer (e.g., periphilar cancer, distal bile duct cancer, intrahepatic bile duct cancer), bladder cancer, bone cancer (e.g., osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g., meningioma, astocytoma, oligodendrogliomas, ependymoma, gliomas, medulloblastoma, ganglioglioma, Schwannoma, germinoma, craniopharyngioma), breast cancer (e.g., ductal carcinoma in situ, infiltrating ductal carcinoma, infiltrating, lobular carcinoma, lobular carcinoma in, situ, gynecomastia), Castleman disease (e.g., giant lymph node hyperplasia, angiofollicular lymph node hyperplasia), cervical cancer, colorectal cancer, endometrial cancer (e.g., endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g., choriocarcinoma, chorioadenoma destruens), Hodgkin's disease, non-Hodgkin's lymphoma, Kaposi's sarcoma, kidney cancer (e.g., renal cell cancer), laryngeal and hypopharyngeal cancer, liver cancer (e.g., hemangioma, hepatic adenoma, focal nodular hyperplasia, hepatocellular carcinoma), lung cancer (e.g., small cell lung cancer, non-small cell lung cancer), mesothelioma, plasmacytoma, nasal cavity and paranasal sinus cancer (e.g., esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g., embryonal rhabdomyosarcoma, alveolar rhabdomyosarcoma, pleomorphic rhabdomyosarcoma), salivary gland cancer, skin cancer (e.g., melanoma, nonmelanoma skin cancer), stomach cancer, testicular cancer (e.g., seminoma, nonseminoma germ cell cancer), thymus cancer, thyroid cancer (e.g., follicular carcinoma, anaplastic carcinoma, poorly differentiated carcinoma, medullary thyroid carcinoma, thyroid lymphoma), vaginal cancer, vulvar cancer, and uterine cancer (e.g., uterine leiomyosarcoma).

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “associated” or “associated with” in the context of a substance or substance activity or function associated with a disease (e.g., cancer) means that the disease is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g., colon cancer, lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, brain cancer, neuroblastoma). For example, certain methods herein treat cancer by decreasing or reducing or preventing the occurrence, growth, metastasis, or progression of cancer; or treat cancer by decreasing a symptom of cancer. Symptoms of cancer (e.g., lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, brain cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g., achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### II. Compounds

In an aspect is provided a compound, or a pharmaceutically acceptable salt thereof, having the formula:

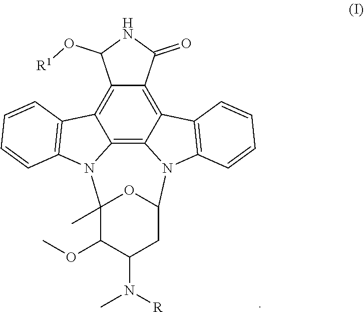

R and R¹ are independently hydrogen, halogen, —CX²₃, —CHX²₂, —CH₂X²,
“*\MERGEFORMAT\* MERGEFORMAT —OCX²₃, —OCHX²₂, —OCH₂X², —CN, —SOₙ₂R², —SOᵥ₂NR²R³, —NR⁴NR²R³, —ONR²R³,
“*\MERGEFORMAT\* MERGEFORMAT —NR⁴C(O)NR²R³, —N(O)ₘ₂, —NR²R³, —C(O)R², —C(NH)R², —C(O)OR², —O C(O)R², —OC(O)OR²,
“*\MERGEFORMAT\* MERGEFORMAT —C(O)NR²R³, —OC(O)NR²R³, —OR², —SR², —NR⁴SO₂R², —NR⁴C(O)R², —N R⁴C(O)OR²,
“*\MERGEFORMAT\*MERGEFORMAT —NR²OR³, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
R², R³, and R⁴ are independently hydrogen, halogen, —CCl₃, —CBr₃, —CF₃, —CI₃,
“*\MERGEFORMAT\* MERGEFORMAT —CHCl₂, —CHBr₂, —CHF₂, —CHI₂, —CH₂Cl, —CH₂Br, —CH₂F, —CH₂I, —CN, —OH, —NH₂, —COOH,
“*\MERGEFORMAT\* MERGEFORMAT —CONH₂, —OCCl₃, —OCF₃, —OCBr₃, —OCI₃, —OCHCl₂, —OCHBr₂, —OCHI₂, —OCHF₂, —OCH₂Cl,
“*\MERGEFORMAT\*MERGEFORMAT —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R² and R³ substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbols m2 and v2 are independently 1 or 2.

The symbol n2 is an integer from 0 to 4.

Each X² is independently —Cl, —Br, —I, or —F.

In embodiments, R and R¹ are not both hydrogen.

In embodiments, the compound has the formula:

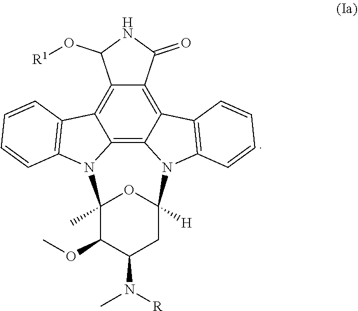

R and R¹ are as described herein, including in embodiments.

In embodiments, a substituted R (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R is substituted with a plurality of groups selected from substituent groups, size-limited substituent groups, and lower substituent groups; each substituent group, size-limited substituent group, and/or lower substituent group may optionally be different. In embodiments, when R is substituted, it is substituted with at least one substituent group. In embodiments, when R is substituted, it is substituted with at least one size-limited substituent group. In embodiments, when R is substituted, it is substituted with at least one lower substituent group.

In embodiments, R is hydrogen, halogen, —CCl₃, —CBr₃, —CF₃, —CI₃, —CH₂Cl,
“*\MERGEFORMAT\* MERGEFORMAT —CH₂Br, —CH₂F, —CH₂J, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H,
“*\MERGEFORMAT\* MERGEFORMAT —NHC(O)H, —NHC(O)OH, —NHOH, —OCCl₃, —OCBr₃, —OCF₃, —OCI₃, —O CH₂Cl, —OCH₂Br,
“*\MERGEFORMAT\* MERGEFORMAT —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R is hydrogen. In embodiments, R is halogen. In embodiments, R is —F. In embodiments, R is —Cl. In embodiments, R is —Br. In embodiments, R is —I. In embodiments, R is —CCl₃. In embodiments, R is —CBr₃. In embodiments, R is —CF₃. In embodiments, R is —CI₃. In embodiments, R is —CH₂Cl. In embodiments, R is —CH₂Br. In embodiments, R is —CH₂F. In embodiments, R is —CH₂I. In embodiments, R is —CHCl₂. In embodiments, R is —CHBr₂. In embodiments, R is —CHF₂. In embodiments, R is —CHI₂. In embodiments, R is —CN. In embodiments, R is —OH. In embodiments, R is —NH₂. In embodiments, R is —COOH. In embodiments, R is —CONH₂. In embodiments, R is —NO₂. In embodiments, R is —SH. In embodiments, R is —SO₃H. In embodiments, R is —OSO₃H. In embodiments, R is —SO₂NH₂. In embodiments, R is —NHNH₂. In embodiments, R is —ONH₂. In embodiments, R is —NHC(O)NHNH₂. In embodiments, R is —NHC(O)NH₂. In embodiments, R is —NHSO₂H. In embodiments, R is —NHC(O)H. In embodiments, R is —NHC(O)OH. In embodiments, R is-NHOH. In embodiments, R is —OCCl₃. In embodiments, R is —OCBr₃. In embodiments, R is —OCF₃. In embodiments, R is —OCI₃. In embodiments, R is —OCH₂Cl. In embodiments, R is —OCH₂Br. In embodiments, R is —OCH₂F. In embodiments, R is —OCH₂I. In embodiments, R is —OCHCl₂. In embodiments, R is —OCHBr₂. In embodiments, R is —OCHF₂. In embodiments, R is —OCHI₂. In embodiments, R is unsubstituted C₁-C₄ alkyl. In embodiments, R is unsubstituted methyl. In embodiments, R is unsubstituted ethyl. In embodiments, R is unsubstituted propyl. In embodiments, R is unsubstituted n-propyl. In embodiments, R is unsubstituted isopropyl. In embodiments, R is unsubstituted butyl. In embodiments, R is unsubstituted n-butyl. In embodiments, R is unsubstituted isobutyl. In embodiments, R is unsubstituted tert-butyl.

In embodiments, R is hydrogen,

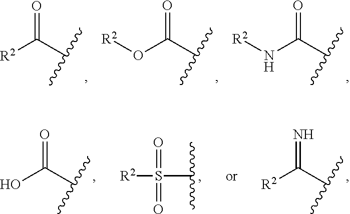

wherein R² is as described herein, including in embodiments. In embodiments, R is hydrogen. In embodiments, R is

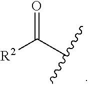

In embodiments, R is

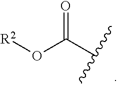

In embodiments, R is

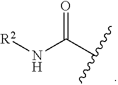

In embodiments, R is

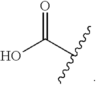 [SMILES: *C(=O)O]

In embodiments, R is

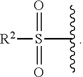

In embodiments, R is

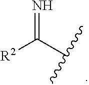

In embodiments, R is hydrogen,

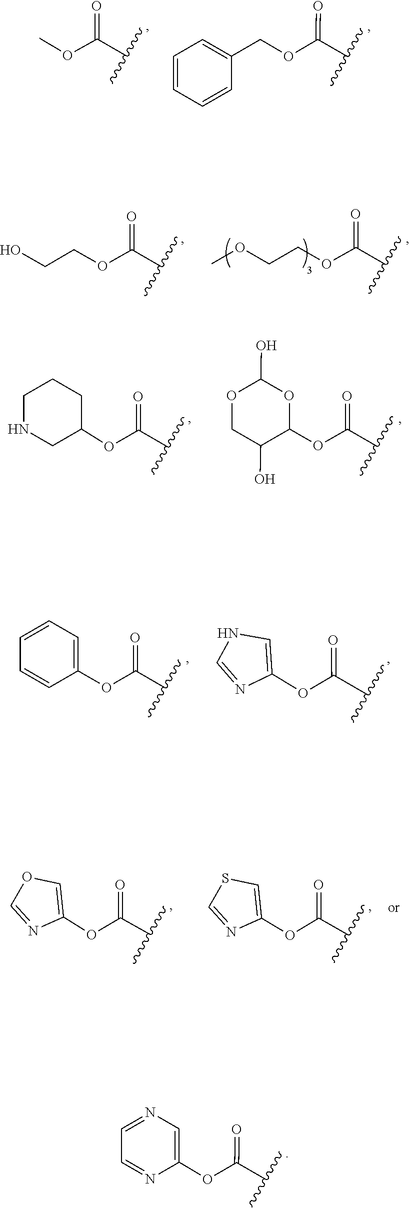

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, halogen, —CCl₃, —CBr₃, —CF₃, —CI₃, —CH₂Cl,
“*\MERGEFORMAT\* MERGEFORMAT —CH₂Br, —CH₂F, —CH₂I, —CHCl₂, —CHBr₂, —CHF₂, —CHI₂, —CN, —OH, —NH ₂, —COOH, —CONH₂, —NO₂, —SH, —SO₃H, —OSO₃H, —SO₂NH₂, —NHNH₂, —ONH₂, —NHC(O)NH₂, —NHSO₂H,
“*\MERGEFORMAT\* MERGEFORMAT —NHC(O)H, —NHC(O)OH, —NHOH, —OCCl₃, —OCBr₃, —OCF₃, —OCI₃, —O CH₂Cl, —OCH₂Br,
“*\MERGEFORMAT\* MERGEFORMAT —OCH₂F, —OCH₂J, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is hydrogen. In embodiments, R¹ is halogen. In embodiments, R¹ is —F. In embodiments, R¹ is —Cl. In embodiments, R¹ is —Br. In embodiments, R¹ is —I. In embodiments, R¹ is —CCl₃. In embodiments, R¹ is —CBr₃. In embodiments, R¹ is —CF₃. In embodiments, R¹ is —CI₃. In embodiments, R¹ is —CH₂Cl. In embodiments, R¹ is —CH₂Br. In embodiments, R¹ is —CH₂F. In embodiments, R¹ is —CH₂I. In embodiments, R¹ is —CHCl₂. In embodiments, R¹ is —CHBr₂. In embodiments, R¹ is —CHF₂. In embodiments, R¹ is —CHI₂. In embodiments, R¹ is —CN. In embodiments, R¹ is —OH. In embodiments, R¹ is —NH₂. In embodiments, R¹ is —COOH. In embodiments, R¹ is —CONH₂. In embodiments, R¹ is —NO₂. In embodiments, R¹ is —SH. In embodiments, R¹ is —SO₃H. In embodiments, R¹ is —OSO₃H. In embodiments, R¹ is —SO₂NH₂. In embodiments, R¹ is —NHNH₂. In embodiments, R¹ is —ONH₂. In embodiments, R¹ is —NHC(O)NHNH₂. In embodiments, R¹ is —NHC(O)NH₂. In embodiments, R¹ is —NHSO₂H. In embodiments, R¹ is —NHC(O)H. In embodiments, R¹ is —NHC(O)OH. In embodiments, R¹ is-NHOH. In embodiments, R¹ is —OCCl₃. In embodiments, R¹ is —OCBr₃. In embodiments, R¹ is —OCF₃. In embodiments, R¹ is —OCI₃. In embodiments, R¹ is —OCH₂Cl. In embodiments, R¹ is —OCH₂Br. In embodiments, R¹ is —OCH₂F. In embodiments, R¹ is —OCH₂I. In embodiments, R¹ is —OCHCl₂. In embodiments, R¹ is —OCHBr₂. In embodiments, R¹ is —OCHF₂. In embodiments, R¹ is —OCHI₂. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl. In embodiments, R¹ is unsubstituted phenyl.

In embodiments, R¹ is hydrogen, unsubstituted C₁-C₄ alkyl, unsubstituted phenyl, or —(CH₂CH₂O)ₙ-(unsubstituted C₁-C₄ alkyl), wherein n is an integer from 1 to 12. In embodiments, R¹ is hydrogen, unsubstituted methyl, unsubstituted butyl, unsubstituted phenyl, or —(CH₂CH₂O)₃—CH₃. In embodiments, R¹ is —(CH₂CH₂O)ₙ—CH₃. In embodiments, R¹ is —(CH₂CH₂O)ₙ—CH₂CH₃. In embodiments, R¹ is —CH₂CH₂OCH₃. In embodiments, R¹ is —(CH₂CH₂O)₂—CH₃. In embodiments, R¹ is —(CH₂CH₂O)₃—CH₃. In embodiments, R¹ is
“*\MERGEFORMAT\*MERGEFORMAT —(CH₂CH₂O)₄—CH₃.

In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12.

In embodiments, R or R¹ is a cleavable moiety. In embodiments, R and R¹ are each a cleavable moiety. In embodiments, the cleavable moiety is

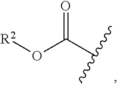

wherein R² is as described herein, including in embodiments.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is independently hydrogen. In embodiments, R² is independently halogen. In embodiments, R² is independently —F. In embodiments, R² is independently —Cl. In embodiments, R² is independently —Br. In embodiments, R² is independently —I. In embodiments, R² is independently —CCl₃. In embodiments, R² is independently —CBr₃. In embodiments, R² is independently —CF₃. In embodiments, R² is independently —CI₃. In embodiments, R² is independently —CH₂Cl. In embodiments, R² is independently —CH₂Br. In embodiments, R² is independently —CH₂F. In embodiments, R² is independently —CH₂I. In embodiments, R² is independently —CHCl₂. In embodiments, R² is independently —CHBr₂. In embodiments, R² is independently —CHF₂. In embodiments, R² is independently —CHI₂. In embodiments, R² is independently —CN. In embodiments, R² is independently —OH. In embodiments, R² is independently —NH₂. In embodiments, R² is independently —COOH. In embodiments, R² is independently —CONH₂. In embodiments, R² is independently —OCCl₃. In embodiments, R² is independently —OCBr₃. In embodiments, R² is independently —OCF₃. In embodiments, R² is independently —OCI₃. In embodiments, R² is independently —OCH₂Cl. In embodiments, R² is independently —OCH₂Br. In embodiments, R² is independently —OCH₂F. In embodiments, R² is independently —OCH₂I. In embodiments, R² is independently —OCHCl₂. In embodiments, R² is independently —OCHBr₂. In embodiments, R² is independently —OCHF₂. In embodiments, R² is independently —OCHI₂. In embodiments, R² is independently unsubstituted C₁-C₄ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted isobutyl. In embodiments, R² is independently unsubstituted tert-butyl. In embodiments, R² is independently substituted C₁-C₄ alkyl. In embodiments, R² is independently —CH₂CH₂OH. In embodiments, R² is independently —CH₂-(unsubstituted phenyl). In embodiments, R² is independently substituted or unsubstituted 3 to 6 membered heterocycloalkyl. In embodiments, R² is independently unsubstituted piperidinyl. In embodiments, R² is independently substituted or unsubstituted dioxanyl. In embodiments, R² is independently substituted dioxanyl. In embodiments, R² is independently

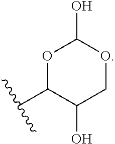 [SMILES: *C1OC(O)OCC1O]

In embodiments, R² is independently

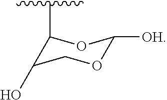 [SMILES: *C1OC(O)OCC1O]

In embodiments, R² is independently

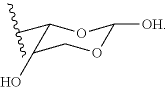 [SMILES: *C1OC(O)OCC1O]

In embodiments, R² is independently unsubstituted dioxanyl. In embodiments, R² is independently unsubstituted phenyl. In embodiments, R² is independently unsubstituted 5 to 6 membered heteroaryl. In embodiments, R² is independently unsubstituted imidazolyl. In embodiments, R² is independently unsubstituted oxazolyl. In embodiments, R² is independently unsubstituted thiazolyl. In embodiments, R² is independently unsubstituted pyrazinyl.

In embodiments, R² is independently hydrogen, substituted or unsubstituted C₁-C₄ alkyl, substituted or unsubstituted 3 to 6 membered heterocycloalkyl, unsubstituted phenyl, unsubstituted 5 to 6 membered heteroaryl, or —(CH₂CH₂O)ₙ-(unsubstituted C₁-C₄ alkyl), wherein n is an integer from 1 to 12. In embodiments, R² is independently hydrogen, unsubstituted methyl, —CH₂CH₂OH, —CH₂-(unsubstituted phenyl), unsubstituted piperidinyl, substituted dioxanyl, unsubstituted phenyl, unsubstituted imidazolyl, unsubstituted oxazolyl, unsubstituted thiazolyl, unsubstituted pyrazinyl, —(CH₂CH₂O)₂—CH₃, —(CH₂CH₂O)₃—CH₃, or
“*\MERGEFORMAT\*MERGEFORMAT —(CH₂CH₂O)₄—CH₃. In embodiments, R² is independently —(CH₂CH₂O)ₙ-(unsubstituted C₁-C₄ alkyl). In embodiments, R² is independently —(CH₂CH₂O)ₙ—CH₃. In embodiments, R² is independently —(CH₂CH₂O)ₙ—CH₂CH₃. In embodiments, R² is independently
“*\MERGEFORMAT\*MERGEFORMAT —CH₂CH₂OCH₃. In embodiments, R² is independently —(CH₂CH₂O)₂—CH₃. In embodiments, R² is independently —(CH₂CH₂O)₃—CH₃. In embodiments, R² is independently
“*\MERGEFORMAT\*MERGEFORMAT —(CH₂CH₂O)₄—CH₃.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is independently hydrogen. In embodiments, R³ is independently halogen. In embodiments, R³ is independently —F. In embodiments, R³ is independently —Cl. In embodiments, R³ is independently —Br. In embodiments, R³ is independently —I. In embodiments, R³ is independently —CCl₃. In embodiments, R³ is independently —CBr₃. In embodiments, R³ is independently —CF₃. In embodiments, R³ is independently —CI₃. In embodiments, R³ is independently —CH₂Cl. In embodiments, R³ is independently —CH₂Br. In embodiments, R³ is independently —CH₂F. In embodiments, R³ is independently —CH₂J. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CN. In embodiments, R³ is independently —OH. In embodiments, R³ is independently —NH₂. In embodiments, R³ is independently —COOH. In embodiments, R³ is independently —CONH₂. In embodiments, R³ is independently —OCCl₃. In embodiments, R³ is independently —OCBr₃. In embodiments, R³ is independently —OCF₃. In embodiments, R³ is independently —OCI₃. In embodiments, R³ is independently —OCH₂Cl. In embodiments, R³ is independently —OCH₂Br. In embodiments, R³ is independently —OCH₂F. In embodiments, R³ is independently —OCH₂I. In embodiments, R³ is independently —OCHCl₂. In embodiments, R³ is independently
“*\MERGEFORMAT\*MERGEFORMAT —OCHBr₂. In embodiments, R³ is independently —OCHF₂. In embodiments, R³ is independently —OCHI₂. In embodiments, R³ is independently unsubstituted C₁-C₄ alkyl. In embodiments, R³ is independently unsubstituted methyl. In embodiments, R³ is independently unsubstituted ethyl. In embodiments, R³ is independently unsubstituted propyl. In embodiments, R³ is independently unsubstituted n-propyl. In embodiments, R³ is independently unsubstituted isopropyl. In embodiments, R³ is independently unsubstituted butyl. In embodiments, R³ is independently unsubstituted n-butyl. In embodiments, R³ is independently unsubstituted isobutyl. In embodiments, R³ is independently unsubstituted tert-butyl.

In embodiments, a substituted ring formed when R² and R³ substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R² and R³ substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R³ substituents bonded to the same nitrogen atom are joined is substituted, it is substituted with at least one substituent group. In embodiments, when the substituted ring formed when R² and R³ substituents bonded to the same nitrogen atom are joined is substituted, it is substituted with at least one size-limited substituent group. In embodiments, when the substituted ring formed when R² and R³ substituents bonded to the same nitrogen atom are joined is substituted, it is substituted with at least one lower substituent group.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is independently hydrogen. In embodiments, R⁴ is independently halogen. In embodiments, R⁴ is independently —F. In embodiments, R⁴ is independently —Cl. In embodiments, R⁴ is independently —Br. In embodiments, R⁴ is independently —I. In embodiments, R⁴ is independently —CCl₃. In embodiments, R⁴ is independently —CBr₃. In embodiments, R⁴ is independently —CF₃. In embodiments, R⁴ is independently —CI₃. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —CHCl₂. In embodiments, R⁴ is independently —CHBr₂. In embodiments, R⁴ is independently —CHF₂. In embodiments, R⁴ is independently —CHI₂. In embodiments, R⁴ is independently —CN. In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —OCCl₃. In embodiments, R⁴ is independently —OCBr₃. In embodiments, R⁴ is independently —OCF₃. In embodiments, R⁴ is independently —OCI₃. In embodiments, R⁴ is independently —OCH₂Cl. In embodiments, R⁴ is independently —OCH₂Br. In embodiments, R⁴ is independently —OCH₂F. In embodiments, R⁴ is independently —OCH₂I. In embodiments, R⁴ is independently —OCHCl₂. In embodiments, R⁴ is independently
“*\MERGEFORMAT\*MERGEFORMAT —OCHBr₂. In embodiments, R⁴ is independently —OCHF₂. In embodiments, R⁴ is independently —OCHI₂. In embodiments, R⁴ is independently unsubstituted C₁-C₄ alkyl. In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted n-propyl. In embodiments, R⁴ is independently unsubstituted isopropyl. In embodiments, R⁴ is independently unsubstituted butyl. In embodiments, R⁴ is independently unsubstituted n-butyl. In embodiments, R⁴ is independently unsubstituted isobutyl. In embodiments, R⁴ is independently unsubstituted tert-butyl.

In embodiments, when R is substituted, R is substituted with one or more first substituent groups denoted by R^{.1} as explained in the definitions section above in the description of “first substituent group(s)”. In embodiments, when an R^{.1} substituent group is substituted, the R^{.1} substituent group is substituted with one or more second substituent groups denoted by R^{.2} as explained in the definitions section above in the description of “first substituent group(s)”. In embodiments, when an R^{.2} substituent group is substituted, the R^{.2} substituent group is substituted with one or more third substituent groups denoted by R^{.3} as explained in the definitions section above in the description of “first substituent group(s)”. In the above embodiments, R, R^{.1}, R^{.2}, and R^{.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R, R^{.1}, R^{.2}, and R^{.3}, respectively.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R″, R^{WW.1}, R^{WW.2}, and R^{WW.3} correspond to R³, R^{3.1}, R^{3.2}, and R³-3, respectively.

In embodiments, when R² and R³ substituents bonded to the same nitrogen atom are optionally joined to form a moiety that is substituted (e.g., a substituted heterocycloalkyl or substituted heteroaryl), the moiety is substituted with one or more first substituent groups denoted by R²¹ as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R² and R³ substituents bonded to the same nitrogen atom are optionally joined to form a moiety that is substituted (e.g., a substituted heterocycloalkyl or substituted heteroaryl), the moiety is substituted with one or more first substituent groups denoted by R³¹ as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R″, R^{WW.1}, R^{WW.2}, and R^{WW.3} correspond to R⁴, R^{4.1}, R^{4.2}, and R^{4.3}, respectively.

In embodiments, the compound has the formula:

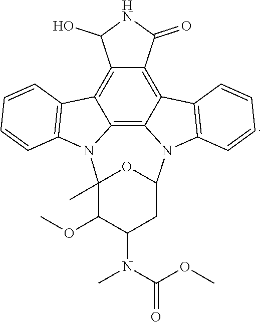 [SMILES: COC(=O)N(C)C1CC2OC(C)(C1OC)n1c3ccccc3c3c4c(c5c6ccccc6n2c5c31)C(=O)NC4O]

In embodiments, the compound has the formula:

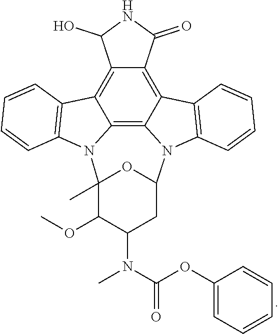 [SMILES: COC1C(N(C)C(=O)Oc2ccccc2)CC2OC1(C)n1c3ccccc3c3c4c(c5c6ccccc6n2c5c31)C(=O)NC4O]

In embodiments, the compound has the formula:

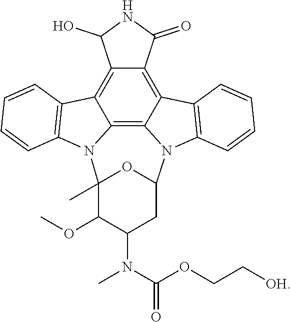 [SMILES: COC1C(N(C)C(=O)OCCO)CC2OC1(C)n1c3ccccc3c3c4c(c5c6ccccc6n2c5c31)C(=O)NC4O]

In embodiments, the compound has the formula:

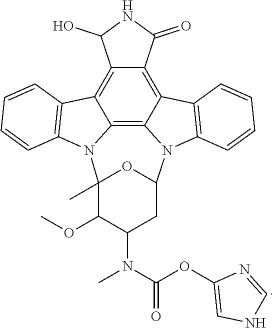 [SMILES: COC1C(N(C)C(=O)Oc2c[nH]cn2)CC2OC1(C)n1c3ccccc3c3c4c(c5c6ccccc6n2c5c31)C(=O)NC4O]

In embodiments, the compound has the formula:

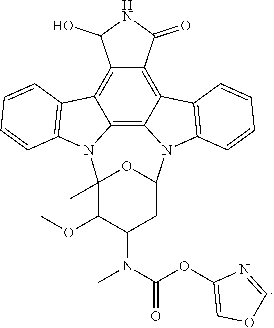 [SMILES: COC1C(N(C)C(=O)Oc2cocn2)CC2OC1(C)n1c3ccccc3c3c4c(c5c6ccccc6n2c5c31)C(=O)NC4O]

In embodiments, the compound has the formula:

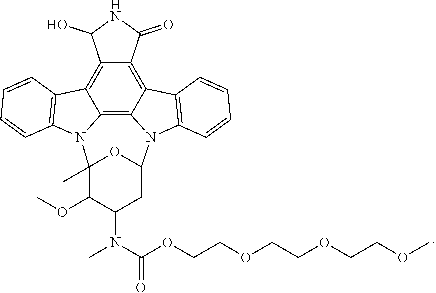 [SMILES: COCCOCCOCCOC(=O)N(C)C1CC2OC(C)(C1OC)n1c3ccccc3c3c4c(c5c6ccccc6n2c5c31)C(=O)NC4O]

In embodiments, the compound has the formula:

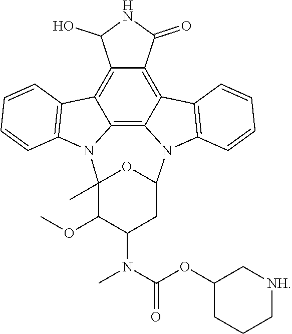 [SMILES: COC1C(N(C)C(=O)OC2CCCNC2)CC2OC1(C)n1c3ccccc3c3c4c(c5c6ccccc6n2c5c31)C(=O)NC4O]

In embodiments, the compound has the formula:

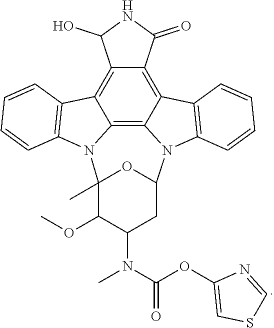 [SMILES: COC1C(N(C)C(=O)Oc2cscn2)CC2OC1(C)n1c3ccccc3c3c4c(c5c6ccccc6n2c5c31)C(=O)NC4O]

In embodiments, the compound has the formula:

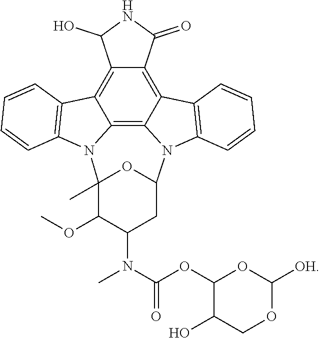 [SMILES: COC1C(N(C)C(=O)OC2OC(O)OCC2O)CC2OC1(C)n1c3ccccc3c3c4c(c5c6ccccc6n2c5c31)C(=O)NC4O]

In embodiments, the compound has the formula:

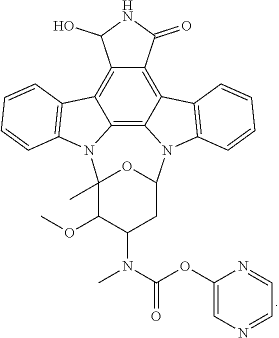 [SMILES: COC1C(N(C)C(=O)Oc2cnccn2)CC2OC1(C)n1c3ccccc3c3c4c(c5c6ccccc6n2c5c31)C(=O)NC4O]

In embodiments, the compound has the formula:

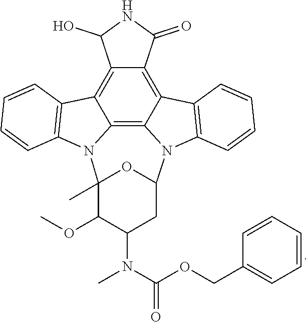 [SMILES: COC1C(N(C)C(=O)OCc2ccccc2)CC2OC1(C)n1c3ccccc3c3c4c(c5c6ccccc6n2c5c31)C(=O)NC4O]

In embodiments, the compound has the formula:

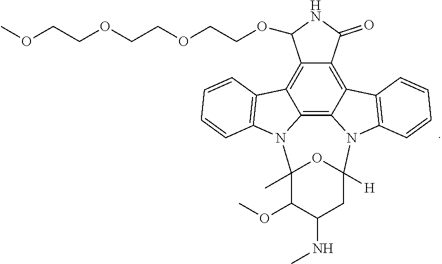 [SMILES: CNC1CC2OC(C)(C1OC)n1c3ccccc3c3c4c(c5c6ccccc6n2c5c31)C(=O)NC4OCCOCCOCCOC]

In embodiments, the compound has the formula:

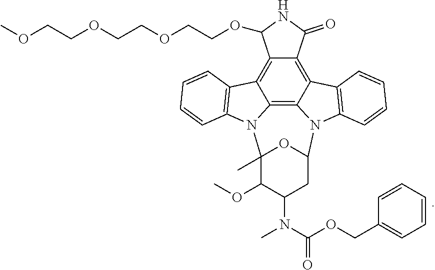 [SMILES: COCCOCCOCCOC1NC(=O)c2c1c1c3ccccc3n3c1c1c2c2ccccc2n1C1CC(N(C)C(=O)OCc2ccccc2)C(OC)C3(C)O1]

In embodiments, the compound has the formula:

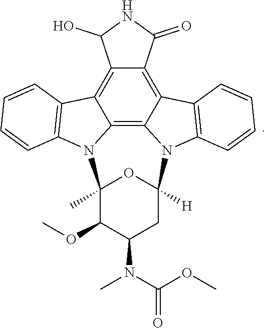 [SMILES: COC(=O)N(C)[C@@H]1C[C@H]2O[C@@](C)([C@@H]1OC)n1c3ccccc3c3c4c(c5c6ccccc6n2c5c31)C(=O)NC4O]

In embodiments, the compound has the formula:

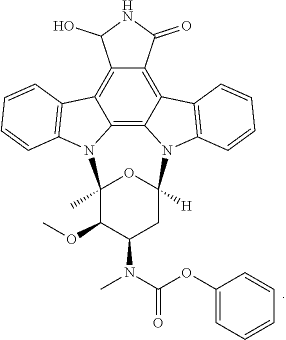 [SMILES: CO[C@@H]1[C@H](N(C)C(=O)Oc2ccccc2)C[C@H]2O[C@]1(C)n1c3ccccc3c3c4c(c5c6ccccc6n2c5c31)C(=O)NC4O]

In embodiments, the compound has the formula:

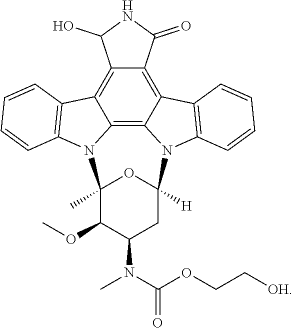 [SMILES: CO[C@@H]1[C@H](N(C)C(=O)OCCO)C[C@H]2O[C@]1(C)n1c3ccccc3c3c4c(c5c6ccccc6n2c5c31)C(=O)NC4O]

In embodiments, the compound has the formula:

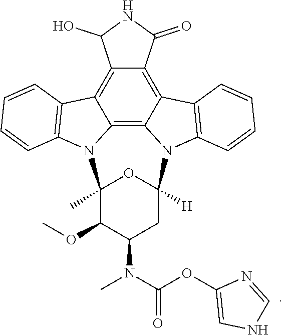 [SMILES: CO[C@@H]1[C@H](N(C)C(=O)Oc2c[nH]cn2)C[C@H]2O[C@]1(C)n1c3ccccc3c3c4c(c5c6ccccc6n2c5c31)C(=O)NC4O]

In embodiments, the compound has the formula:

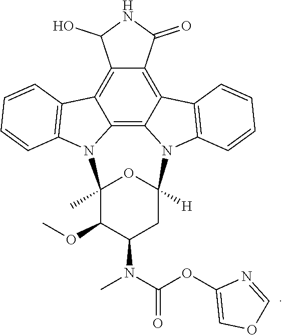 [SMILES: CO[C@@H]1[C@H](N(C)C(=O)Oc2cocn2)C[C@H]2O[C@]1(C)n1c3ccccc3c3c4c(c5c6ccccc6n2c5c31)C(=O)NC4O]

In embodiments, the compound has the formula:

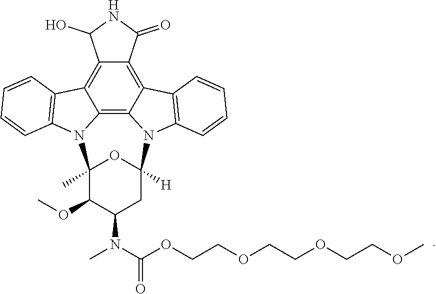 [SMILES: COCCOCCOCCOC(=O)N(C)[C@@H]1C[C@H]2O[C@@](C)([C@@H]1OC)n1c3ccccc3c3c4c(c5c6ccccc6n2c5c31)C(=O)NC4O]

In embodiments, the compound has the formula:

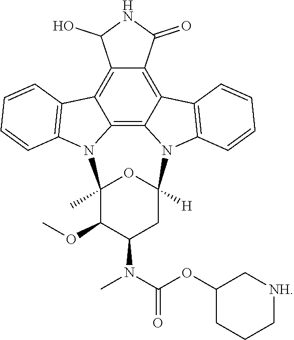 [SMILES: CO[C@@H]1[C@H](N(C)C(=O)OC2CCCNC2)C[C@H]2O[C@]1(C)n1c3ccccc3c3c4c(c5c6ccccc6n2c5c31)C(=O)NC4O]

In embodiments, the compound has the formula:

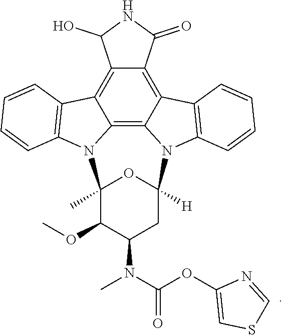 [SMILES: CO[C@@H]1[C@H](N(C)C(=O)Oc2cscn2)C[C@H]2O[C@]1(C)n1c3ccccc3c3c4c(c5c6ccccc6n2c5c31)C(=O)NC4O]

In embodiments, the compound has the formula:

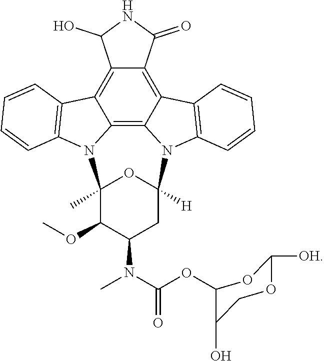 [SMILES: CO[C@@H]1[C@H](N(C)C(=O)OC2OC(O)OCC2O)C[C@H]2O[C@]1(C)n1c3ccccc3c3c4c(c5c6ccccc6n2c5c31)C(=O)NC4O]

In embodiments, the compound has the formula:

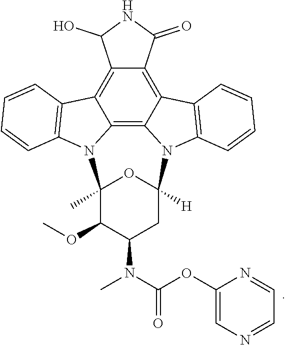 [SMILES: CO[C@@H]1[C@H](N(C)C(=O)Oc2cnccn2)C[C@H]2O[C@]1(C)n1c3ccccc3c3c4c(c5c6ccccc6n2c5c31)C(=O)NC4O]

In embodiments, the compound has the formula:

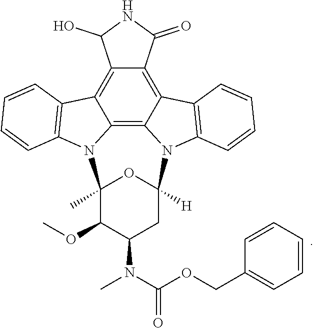 [SMILES: CO[C@@H]1[C@H](N(C)C(=O)OCc2ccccc2)C[C@H]2O[C@]1(C)n1c3ccccc3c3c4c(c5c6ccccc6n2c5c31)C(=O)NC4O]

In embodiments, the compound has the formula:

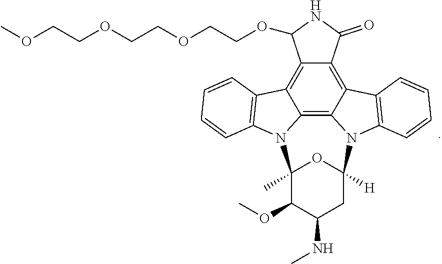 [SMILES: CN[C@@H]1C[C@H]2O[C@@](C)([C@@H]1OC)n1c3ccccc3c3c4c(c5c6ccccc6n2c5c31)C(=O)NC4OCCOCCOCCOC]

In embodiments, the compound has the formula:

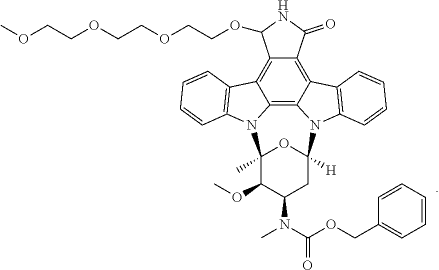 [SMILES: COCCOCCOCCOC1NC(=O)c2c1c1c3ccccc3n3c1c1c2c2ccccc2n1[C@H]1C[C@@H](N(C)C(=O)OCc2ccccc2)[C@@H](OC)[C@]3(C)O1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound or a stable micelle comprising a compound disclosed herein and a pharmaceutically acceptable excipient.

A pharmaceutical composition including a compound or a stable micelle comprising a compound as described herein can be administered by a variety of methods known in the art. The route and/or mode of administration vary depending upon the desired results. In embodiments, administration is intravenous, intramuscular, intraperitoneal, or subcutaneous, or administered proximal to the site of the target. Pharmaceutically acceptable excipients can be suitable for intravenous, intramuscular, subcutaneous, parenteral, spinal or epidermal administration (e.g., by injection or infusion).

Pharmaceutical compositions of the compound or stable micelle comprising a compound can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the compound or stable micelle comprising a compound is employed in the pharmaceutical compositions of the invention. The compound or stable micelle comprising a compound provided can be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It may be advantageous to formulate the compound or stable micelle comprising a compound in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of compound or stable micelle comprising a compound calculated to produce the desired therapeutic effect in association with the required pharmaceutical excipient.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular compound or stable micelle comprising a compound being employed, the duration of the treatment, other drugs, compounds and/or materials used in combination with the particular compositions employed, the age, sex, weight, condition, general health and prior medical history of the patient being treated, and like factors.

In an aspect is provided a composition including a stable micelle comprising a compound disclosed herein.

In an aspect, the compound is a staurosporine analog. In embodiments, the stable micelle includes a polyethylene glycol (PEG) moiety. In embodiments, the stable micelle includes a conjugated block copolymer. Exemplary micelles are described in U.S. Pat. No. 10,220,026. In an aspect, the micelle is a pH-sensitive micelle.

In an aspect, the drug/lipid ratio is 0.09.

In embodiments, the stable micelle includes a staurosporine analog of the formula:

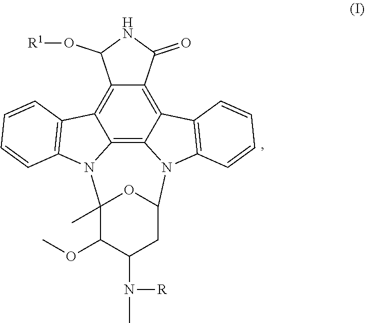

wherein R and R¹ are any chemical group. In embodiments, R or R¹ is

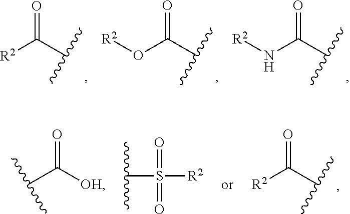

wherein R² is H, an aliphatic hydrocarbon from C₁₋₁₂, an aromatic group such as a phenyl or a heterocyclic group including five and six membered rings with heteroatoms O, N, S or a pegylated compound (CH₂—CH₂—O—)ₙ with n=1 to 12.

In embodiments, the staurosporine analog is 2-methoxyethyl (14-hydroxy-8-methoxy-9-methyl-16-oxo-6,7,8,9,15,16-hexahydro-5H, 14H-17-oxa-4b,9a,15-triaza-5,9-methanodibenzo[b,h]cyclonona[jkl]cyclopenta[e]-as-indacen-7-yl)(methyl)carbamate. In embodiments, the staurosporine analog has the formula:

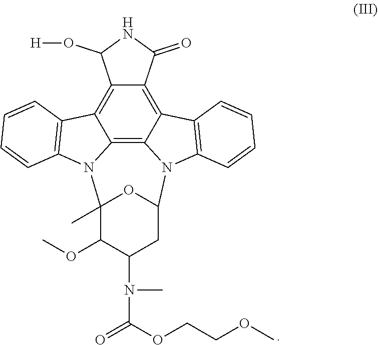 [SMILES: COCCOC(=O)N(C)C1CC2OC(C)(C1OC)n1c3ccccc3c3c4c(c5c6ccccc6n2c5c31)C(=O)NC4O]

In embodiments, the staurosporine analog is 8-methoxy-9-methyl-7-(methylamino)-16-oxo-6,7,8,9,15,16-hexahydro-5H,14H-17-oxa-4b,9a,15-triaza-5,9-methanodibenzo[b,h]cyclonona[jkl]cyclopenta[e]-as-indacen-14-yl 2-phenylacetate. In embodiments, the staurosporine analog has the formula:

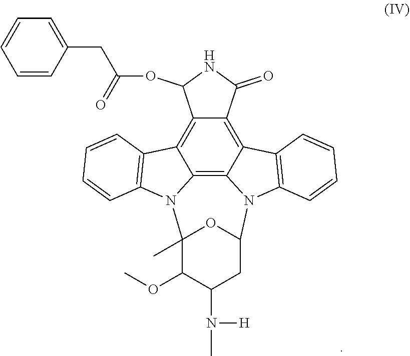 [SMILES: CNC1CC2OC(C)(C1OC)n1c3ccccc3c3c4c(c5c6ccccc6n2c5c31)C(=O)NC4OC(=O)Cc1ccccc1]

In embodiments, the staurosporine analog is 8-methoxy-9-methyl-7-(methylamino)-14-((2-nitro-1H-imidazol-5-yl)oxy)-6,7,8,9,14,15-hexahydro-5H,16H-17-oxa-4b,9a,15-triaza-5,9-methanodibenzo[b,h]cyclonona[jkl]cyclopenta[e]-as-indacen-16-one. In embodiments, the staurosporine analog has the formula:

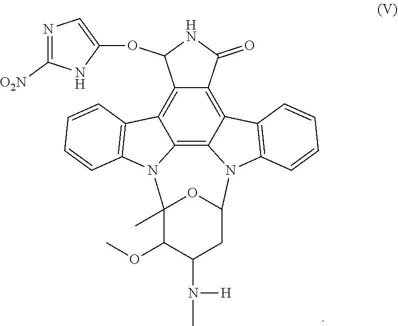 [SMILES: CNC1CC2OC(C)(C1OC)n1c3ccccc3c3c4c(c5c6ccccc6n2c5c31)C(=O)NC4Oc1cnc([N+](=O)[O-])[nH]1]

In embodiments, the staurosporine analog is N-(16-hydroxy-6-methoxy-5-methyl-14-oxo-4a,6,7,8,9,15,16,16c-octahydro-5H,14H-17-oxa-4b,9a,15-triaza-5,9-methanodibenzo[b,h]cyclonona[jkl]cyclopenta[e]-as-indacen-7-yl)-N-methylacetimidamide. In embodiments, the staurosporine analog has the formula:

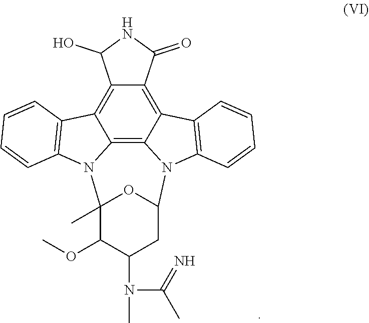 [SMILES: COC1C(N(C)C(C)=N)CC2OC1(C)n1c3ccccc3c3c4c(c5c6ccccc6n2c5c31)C(=O)NC4O]

In embodiments, the staurosporine analog is 7-hydroxystaurosporine (UCN-01) having the formula:

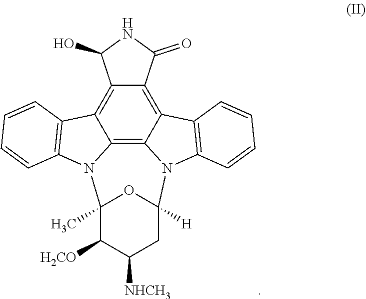

In embodiments, the PEG moiety is covalently attached to R or R¹. In embodiments, the PEG moiety further includes a linker.

### IV. Methods of Treatment

The compounds and pharmaceutical compositions provided herein, including embodiments thereof, are contemplated as providing effective treatments for a disease in a subject such as cancer.

In an aspect is provided a method of treating cancer in a subject in need thereof, the method including administering to a subject a therapeutically effective amount of a compound or a pharmaceutical composition as provided herein including embodiments thereof, thereby treating cancer in the subject.

In an aspect, the compounds and pharmaceutical compositions is administered once. In an aspect, the compounds and pharmaceutical compositions is administered twice. In an aspect, the compounds and pharmaceutical compositions is administered at least two times.

In an aspect, the cancer is a solid tumor. In an aspect, the cancer comprises head and neck cancer (HNSCC), breast cancer, colon cancer, or brain cancer. In an aspect, the cancer is HNSCC. In an aspect, the cancer is breast cancer. In an aspect, the cancer is colon cancer. In an aspect, the cancer is brain cancer. In an aspect, the brain cancer is glioblastoma (GBM). In an aspect the subject is a human.

In an aspect is provided a method of treating a subject with cancer, said method including administering an effective amount of the composition as provided herein including embodiments thereof and administering a chemotherapy or radiotherapy. In an aspect the chemotherapy is doxycycline.

In an aspect, the chemotherapy or radiotherapy is administered concurrently with the compounds and pharmaceutical compositions provided herein. In an aspect, the chemotherapy or radiotherapy is administered after the compounds and pharmaceutical compositions provided herein are administered. In an aspect, the chemotherapy or radiotherapy is administered before the compounds and pharmaceutical compositions provided herein are administered.

### V. Examples

### Example 1: UCN-01 Potency and Pharmacokinetics

UCN-01 potency. The potency of UCN-01 markedly exceeds that of standard GBM agents in GBM U87 cells (Table 1), and it engages a unique pattern of signaling pathways to drive apoptosis. The ability of UCN-01 at noncytotoxic doses to abrogate G2 arrest is evident in p53 dysregulated cells. p53 is mutated in about 35% of primary GBM and 65% of secondary GBM. p53 pathway dysregulation occurs in 86% of GBM.

TABLE 1
GBM agent potency

| Compound | U87 GBM (IC₅₀ nM) |
|---|---|
| UCN-01** | 50 |
| Doxorubicin | 1000 |
| Temozolomide | 50,000 |
| BCNU | 60,000 |
| 5-FU | 2500 |

TABLE 2
Percent unbound fraction of UCN-01 in plasma of different species

| Species | Unbound fraction % |
|---|---|
| Human | <0.02 |
| Mouse | 1.14 |
| Rat | 1.75 |
| Dog | 0.49 |

UCN-01 pharmacokinetics and nanoliposomes. Free UCN-01 is instantly bound by human alpha-1 acid glycoprotein (AAG) plasma protein with extremely high affinity and remains in circulation for remarkably extended periods of time. Table 2 shows the in vitro percent unbound fraction of 1 g/mL of UCN-01 in plasma according to species. Unfortunately this greatly diminishes the availability of free (active) compound so that the in vivo effectiveness of UCN-01 is compromised even when it enters tumor tissue when bound to AAG the UCN-01 molecule is not bioavailable. Increasing the UCN-01 dose to sufficiently saturate AAG in the plasma elevates the risk of toxicity. This has proved problematic in clinical trials and the NIH abandoned further patient testing of UCN-01.

### Example 2: GBM Cytotyoxicity and Radiosensitization with UCN-01

GBM cytotoxicity with UCN-01. 8 different GBM patient cancer stem cell derived GBM tumorsphere lines were incubated with a range of UCN-01 doses. The percent viable cells were determined with the MTT assay. Almost all cell lines had IC50 values in the low nanomolar (nM) range.

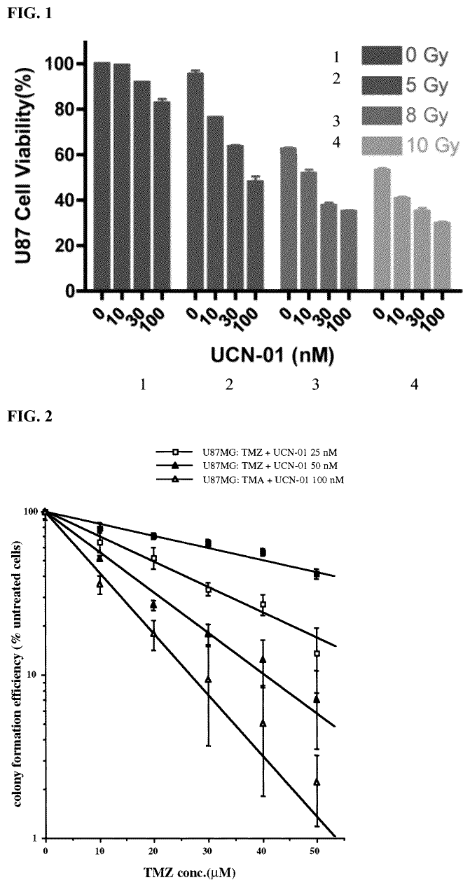

GBM radiosensitization with low doses of the staralog UCN-01. The preliminary data shows that even mildly cytotoxic nanoMolar (nM) levels of UCN-01 (FIG. 1) were able to sensitize radioresistant U87 GBM cells in culture to clinically relevant radiation doses. Moreover, the liposomal loading efficiency of UCN-01 was markedly increased beyond that previously reported, from 25% to 65%. As a precursor to studies with a range of staralogs, extensive model investigations with the class parent staralog model system, STS were performed.

Liposomal loading of UCN-01. The class parent STS and UCN-01 were lipid encapsulated, and these liposomes were taken up by GBM cells and kill them. Initially, for UCN-01 the loading was increased about two times (>50% vs. 25%) beyond that previously reported. Previous pH gradient methods were modified to pH gradient reversal, viz., neutral internal buffer pH and low external buffer pH. Best results were obtained with ammonium-based internal buffers and a drug/lipid ratio of 0.09. Staralog loading efficiencies were >95%.

### Example 3: Staralog Model System

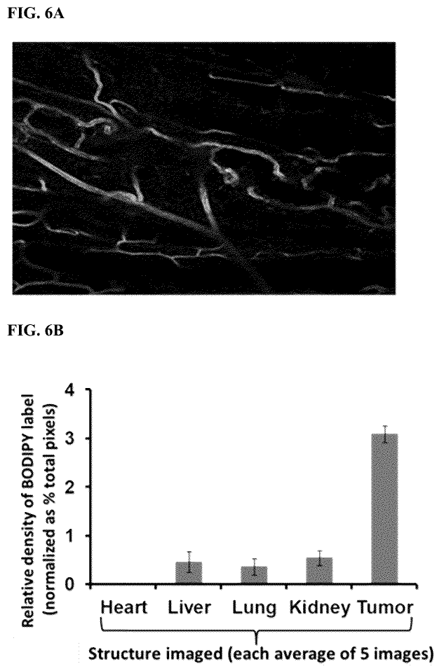

Cytotoxicity Mechanisms of LSTS. Effective cell killing of a range of established and primary GBM lines, and breast and prostate cell lines, with liposomally encapsulated STS (LSTS) was shown. The IC50 values ranged from 1-5 nM and were very close between free and liposomal STS. There was no apparent mechanistic difference in cell death induced in vitro by LSTS versus free STS; several lines of evidence indicated that both induced apoptosis (FIGS. 5A-5C).

STS inhibits the PI-3-kinase mediated phosphorylation of Akt, a key signaling node, and Western blot analysis confirmed suppression of phospho-Akt by both the liposomal and free STS. STS stimulated caspase 3 dependent PARP cleavage to induce apoptosis (FIG. 5A). Apo-one assay results indicated increased caspase 3/7 activity and PARP cleavage aligned with this finding (FIG. 5B). FACS analysis for DNA content of cells treated with both free and liposomal STS showed a G0 shift consistent with apoptosis (FIG. 5C).

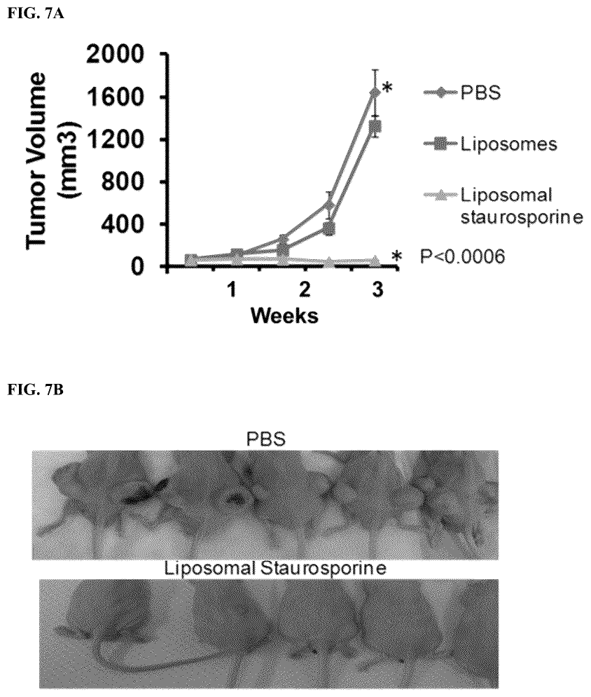

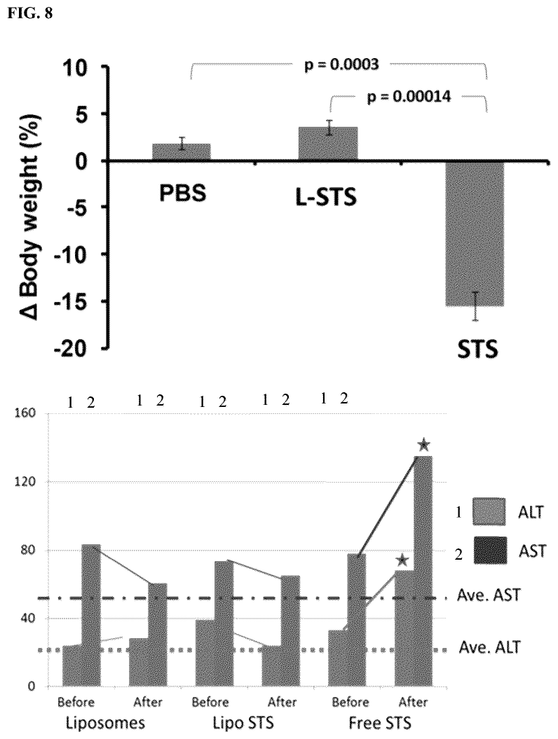

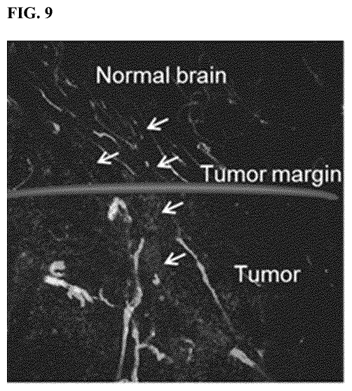

Accumulation of LSTS to suppress flank tumors without apparent toxicity. FIG. 6A depicts liposomal accumulation of LUCN-01 when fluorescent liposomes loaded with STS were injected intravenously in mice. FIG. 6B shows the quantitation for each listed organ from which five slices were acquired and imaged. The highest relative density of liposomes was recorded in the tumor tissue. 0.8 mg/kg of liposomal STS (equivalent in vitro dose: 40 nM) at 3 times/week for 3 weeks caused 100% growth suppression (FIGS. 7A-7B). LSTS suppressed in vivo GBM proliferation (Ki-67) while free STS did not, indicating accumulation at the tumor and a therapeutic effect. This is key because like UCN-01, free STS binds plasma proteins so it is not active at tumors. FIG. 8 shows that animals treated with LSTS did not show a reduction in body weight as was the case with free STS and s subsequent study in which mice were dosed with STS or LSTS (0.8 mg/kg) 3 times per week for two weeks involved terminal liver function tests and gross organ morphology. AST/ALT liver enzymes were elevated in the free STS group, but not the LSTS group.

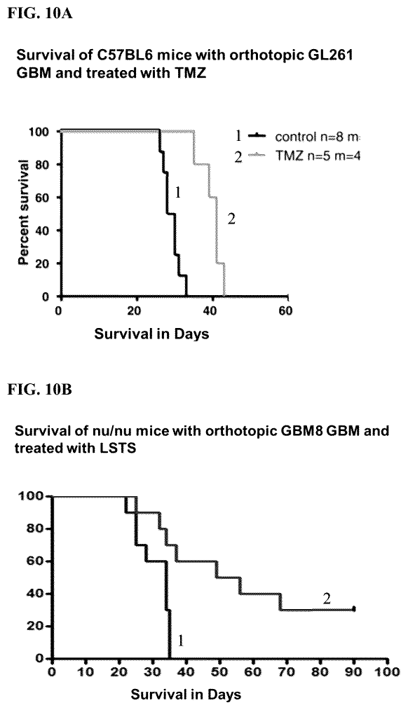

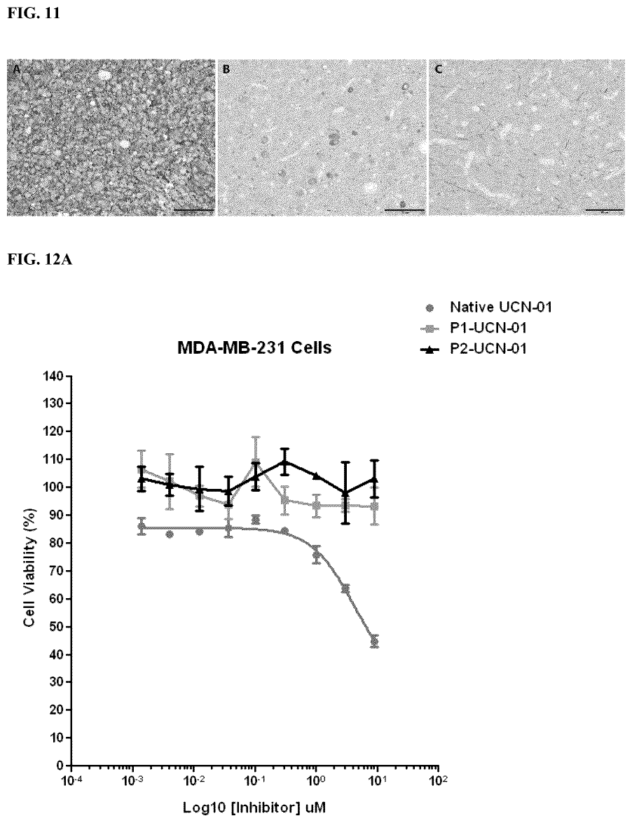

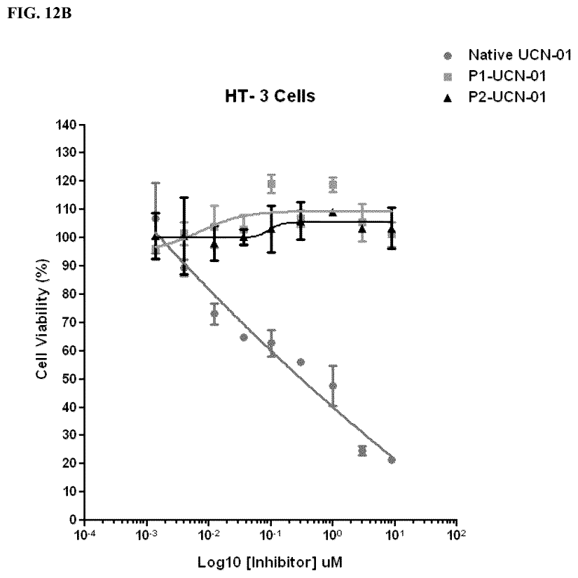

Liposomes loaded with the staralog class parent STS entered orthotopic invasive GBM tumors and local normal brain and markedly extended host survival. Twenty mice were orthotopically inoculated in the right cortex with a GBM cancer stem-like cell line (GBM8) which established brain tumors emulating the invasive natural history of human GBM26. Mice were treated with empty liposomes (control) or LSTS. Starting one week after tumor implantation mice were treated 3× per week for 3 weeks with 0.8 mg/kg LSTS. FIG. 9 clearly indicates that the fluorescently labeled avB3 targeted liposomes bearing STS entered the tumor microvasculature and the tumor parenchyma. Liposomal fluorescence was observed beyond the gross tumor margin, which were found to have porous vessels. FIG. 10 is the Kaplan Meier plot for these treated mice compared to the front line agent TMZ, and reveals that by 60 days post-tumor inoculation, 40% of the LSTS group was alive (p<0.05) and 30% of subjects were alive at 90 days. All controls died by 35 days and in all mice the cause of death was a brain tumor. FIGS. 11A-11C shows that human GBM stains heavily for avB3 compared to normal brain.

### Example 4: UCN-01 Encapsulation

Drug Encapsulation. Briefly, liposomal data for the staralog class parent compound, STS was acquired. Liposomal particle sizes of 50-200 nm and steric stabilization of the liposomal surface by a polyethyleneglycol (PEG)-conjugated lipid reduces contact with both normal tissue and with plasma proteins, and facilitates accumulation in tumor tissue via enhanced permeation and retention (EPR). Liposomal encapsulation also enhances tumor cell drug uptake because liposomes are internalized via endocytosis and bypass drug efflux mechanisms. Liposomal uptake can be accelerated with ligands to internalizing cell surface receptors such as avB3, expressed by GBM tumor cells and microvessels. However when UCN-01 is added to liposomes leakage occurs. Liposomes having a diameter of 120 nm or greater have been formulated to reduce leakage, but these are too large and dilute the drug.

Micelles are a modified form of liposomes and exhibit superior payload loading, size control, and versatility. A micelle is an aggregate of surfactant phospholipid molecules forming a colloidal suspension in a liquid. Micelles are more stable than liposomes and they are much smaller improving tissue penetration. Micelles may also be administered orally while liposomes must be given intravenously. UCN-01 has been loaded into liposomes but leakage has been a problem but the liposomes have to be large and the drug is diluted. Leakage from micelles has been a problem a technique was developed by which to avoid leakage. In circulation the micelles do not dilute the drug because they are small and they are completely nontoxic. If micelles are stable in circulation the compound is delivered and released at multiple sites, but this is not an issue because UCN-01 is biochemically selective for tumors. And the radiation beam is directed only at the tumor and not elsewhere. The micellular formulation is used for three purposes:
1) deliver UCN-01 to sensitize tumors to radiation and chemotherapeutics;
2) deliver UCN-01 to promote healing of tissues by promoting stem/progenitor cell differentiation to neuronal lineage; and
3) promote the differentiation cancer stem cells to a non-progenitor/stem cell phenotype as a means of suppressing tumor growth and expansion.

Method of Fabrication and Composition. Block-copolymer of polyethylene/polypropylene glycol with different molar ratios are used to produce micelles in the 10-30 nm size range. The micelles are water soluble and can be used for both IV and PO administration in tumor models. Size determination of the micelles are done via dynamic light scattering and the physical stability of the micelles are tested by HPLC/Mass spectrometry. PEG-polyethylene copolymers are used to make micelles. The advantages are low toxicity, stability and reduced leakage. The polymeric shell is protective and stable.

### Example 5: UCN-01 Activity in HNSCC

This example shows the treatment of HNSCC with a combination therapy of tumor targeted micelles loaded with UCN-01 in mice and radiation therapy.

Briefly, UCN-01 is loaded into actively tumor targeting micelles to shield UCN-01 from AAG. Due to its small size micellular UCN-01 (mUCN-01) may potentially enter various structures including locoregional lymphatics to selectively radiosensitize resident tumor cells and micrometastases. Intravenously injected mUCN-01 in mice with an HNSCC tumor can enhance HNSCC radioresponse and be well tolerated.

This is based on preliminary data showing, (1) UCN-01 radiosensitization of tumor cells in vitro, (2) the demonstrated tolerability of UCN-01 in patients, and FDA approval for patient testing, (3) the ability of core shell micelles to effectively solubilize a variety of poorly soluble pharmaceutical agents, and (4) micellular biocompatibility, longevity, high stability in vitro and in vivo and the ability to accumulate in tumors via dysfunctional vasculature and active targeting.

### Example 6: UCN-01 and UCN-01 Prodrug in vitro Activity in Breast Cancer Models

This example shows the activity of UCN-01 and UCN-01 prodrugs in breast cancer models.

UCN-01 prodrugs are synthesized according to Example 8 and these are then tested in vitro for hAAG binding, and for efficacy in cultured breast cancer (BC) cells.

hAAG Binding

Each of the hydrolytically stable analogs is analyzed to determine its affinity to human al-acid glycoprotein (hAAG). In the assay, a fixed concentration of drug candidate is incubated with varying concentrations of hAAG immobilized on silica beads (Solvicell, available from XpressBio). Compound concentration unbound to the beads is measured using LC/MS/MS. Compounds with reduced hAAG binding compared to UCN-01 is tested in a cellular efficacy assay.

### Test the Prodrugs Against a Single, UCN-01 Sensitive Cell Line

A triple negative breast cancer line (MDA-MB-231) and normal bovine endothelial cells is cultured in media spiked with hAAG, and treated with native or modified UCN-01 at concentrations between 0.25 nM and 250 nM (in 1-fold drug increments) with or without DOX or radiation therapy (RT). Cellular uptake of UCN-01 is determined via HPLC of lysed cell contents. Genetic profiling reveals p53 mutations, and the levels of phosphorylated CHK1 and Cdc25c, and the signaling hubs PKC, PI3K/Akt, mTOR, Jak/Stat, in each cell line. BC chemoradiosensitization by UCN-01, the UCN-01 prodrug candidates, and vehicle, is quantified and compared with triple negative MDA-MB-231 BC cells in multi-well plates.

### Analog Stability in Serum/Plasma

Compounds with reduced hAAG binding versus UCN-01 advance to stability studies in serum, plasma, and cell media. Some analogs are expected to be stable to serum enzymes and others may rapidly convert to the parent UCN-01. Compounds that rapidly convert exert their biological activity as UCN-01, while compounds that undergo slow or no conversion require the analog itself to have inherent conversion activity. Analogs with divergent stability in serum versus cell media must be interpreted carefully to have confidence that the in vitro cell activity is recapitulated by an in vivo efficacy experiment. Analogs are incubated in human plasma following the Wyeth high-throughput plasma stability assay. Compounds are added to plasma for 2, 5, 15, 60, and 180 minutes. The assay is stopped by addition of cold acetonitrile and precipitated protein removed by centrifuge. Drug concentration in the supernatant is determined by liquid chromatography-mass spectrometry (LC-MS/MS) using a Schimadzu UPLC and SciEx 6500+ mass spectrometer (LC-MS/MS).

### Analog Stability Under the Conditions of the Cell Assay

Analog stability studies are performed in similar manner as described in above, except that serum is replaced by the media used for the cellular efficacy assay.

For Active Analog Compounds with High Stability in Human Serum/Plasma

### Permeability

The cellular permeability of the analogs is measured using the well-established Caco 2 assay. Briefly, compounds are added to Caco-2 monolayers grown on a permeable filter support. Samples are taken from the apical side following basolateral compound addition and from the basolateral side following apical addition at 4 time points. Compound concentrations are determined by LC-MS/MS.

### Activity in Other Breast Cancer Cell Lines

Cytotoxicity of the analogs against MDA-MB-468 cells tested above, along with MDA-MB-231 and MCF-7 cells, is assessed using a CellTiter-Glo (CTG) luminescent cell viability assay. CTG is a homogeneous “add-mix-measure” method of determining the number of viable cells in culture based on quantitation of the ATP present, an indicator of metabolically active cells. This robust protocol has been successfully deployed to measure cytotoxicity in a wide number of cell lines. Briefly, each cell line is optimized for standard assay parameters (e.g., cell seeding density, DMSO sensitivity, optimal endpoint detection time, variance (% CV) using the UCN-01 positive control. Once established, compounds are tested in triplicate 16-point dose response curves in the bovine endothelial cells and human TNBC cells.

### Liver Microsome/Hepatocyte (In Vitro) Stability

In vitro clearance rates in hepatocytes and/or microsomes are often strong predictors of in vivo clearance. UCN-01 is tested to determine whether microsomes or hepatocytes correlate to the published in vivo clearance. Then, to triage for in vivo studies, in vitro assays are conducted on the most promising new compounds. Compounds are incubated for 0, 5, 15 and 30 minutes in either hepatocytes or microsomes. After the given time, acetonitrile is added. After centrifugation, the supernatant LC-MS/MS is used to determine compound concentration.

### Example 7: UCN-01 and UCN-01 Prodrug Entry and Sensitization in Orthotopic BC Tumors In Vivo

This example describes the determination of the entry and sensitization activity of UCN-01 and UCN-01 prodrugs in breast cancer models.

### Pharmacokinetics:

Briefly, Balb/c mice (n=3) are dosed at 7.5 mg/kg by intraperitoneal (IP) injection with UCN-01 or prodrug. The dose is known to be efficacious in vivo against multiple breast cancer lines. Terminal blood samples are withdrawn via cardiac puncture under anesthesia at 0.5, 1, 2, 4, and 10 hours. The animals are perfused with cold buffered formalin and the major organs are removed. The individual blood samples are used for hematologic evaluation, chemistry analysis, and liver enzymes. Samples of brain, heart, liver, kidney, spleen, lung, stomach, ileum, colon, sternum, and femur are processed for histopathology. Tissue samples are homogenized and organically extracted to determine UCN-01 or prodrug concentration. Tissue homogenates is extracted by addition of acetonitrile followed by centrifugation and supernatant collection. Drug concentrations are quantified by LC-MS/MS. The organ distribution of free UCN-01 and prodrug-UCN-01 is compared.

### Plasma Protein Binding:

Hydrolytically stable compounds are analyzed for their plasma free fraction to determine free drug concentrations. Comparing free drug concentrations rather than total concentrations is required to determine superiority of the analogs over UCN-01. The free fraction and other ADME parameters for UCN-01 are published. Briefly, compound and plasma samples are preincubated and added to the receiver wells of a rapid equilibrium dialysis (RED) device. Compound concentrations from both the donor and receiver wells are determined by LC-MS/MS. Free fraction in specific organs/tissues or tumor cells are conducted as required for the analyses using similar, well-established methods.

### Efficacy in Triple Negative BC Line Orthotopically Implanted in Mammary Fat Pad

For efficacy testing in vivo a human triple negative BC cell line (MDA-MB-468) is implanted in the mammary fat pad of nu/nu mice. The growth of the tumors and body weight is acquired before tumor inoculation, before treatment, and every second day for three weeks after treatment with the following: vehicle, Vehicle+DOX, Vehicle+radiation (XRT), UCN-01, UCN-01+DOX, UCN-01+XRT, prodrug, prodrug+DOX, prodrug+XRT. UCN-01 or prodrug is injected IP at 7.5 mg/kg derived from Koh et al., (2002), or a more appropriate dose as determined by our PK studies. Animals are dosed on days 5-10 alone, or with doxorubicin, or with only day of 5 Gy radiation exposure of the mammary fat pad. At the end of the monitoring period mice are sacrificed and the tumors dissected free and weighed. Tumors are dissociated and measures for p53, p21, PIK/Akt/mTOR, Jak2/Stat3, and EGFR levels. The mice are dissected for gross pathology, viz., tumor size and extent, and major organs including brain, heart, lung, lung spleen, bone marrow, liver, and GI tract are sectioned and stained, and metastasis and any abnormalities are identified and counted. In one cohort of mice intratumoral levels of UCN-01 and the prodrug are measured via LC-MS/MS using a SciEX 6500+ mass spectrometer.

### Statistical Analysis for Animal Experiments:

In order to demonstrate differences in organ toxicity or tumor endpoint outcomes (size and degree of invasion as a percent of primary volume) the UCN-01 and prodrug groups among mice, Jonckheere-Terpstra rank-based trend tests are used for continuous outcomes and Cochran-Armitage trend tests for binary outcomes to test for a trend with respect to the subgroups. Sample size and power. With 10 mice per group, considering the comparison using targeting and the primary tumor response between treated groups, i.e., tumor volume, using a two-sided t-test, at a type I error of 0.05, there is 80% power to detect a mean difference of 1.4 standard deviations of tumor volumes.

### Example 8: Synthetic Procedures

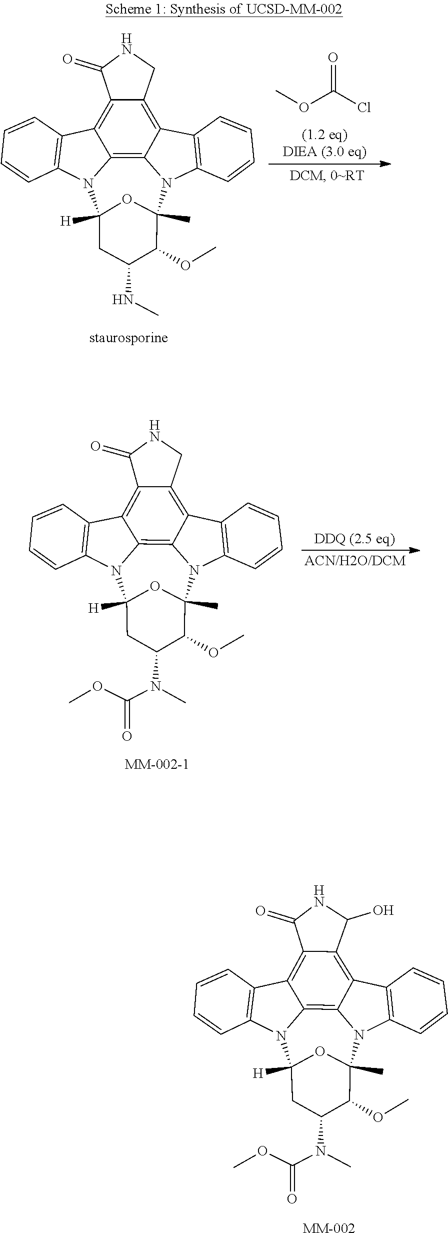

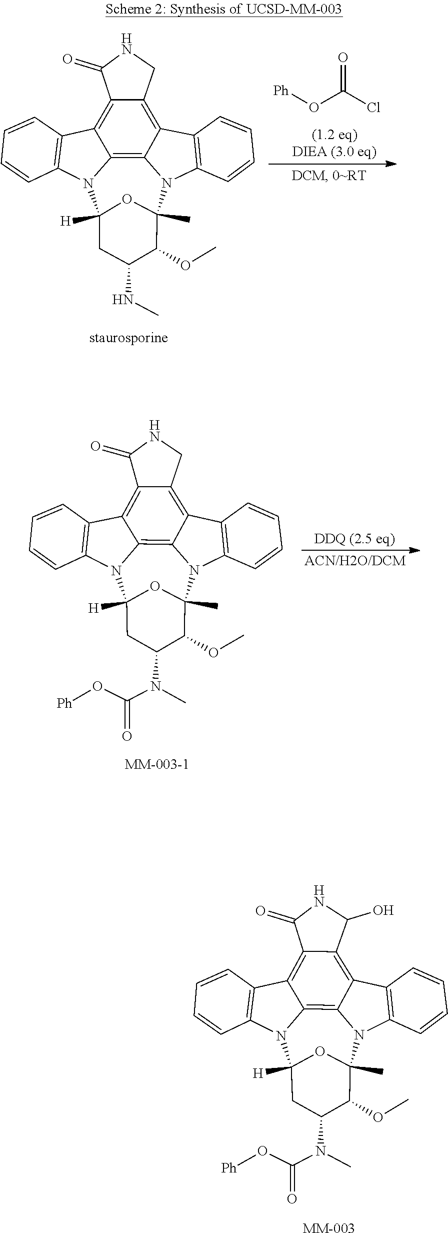

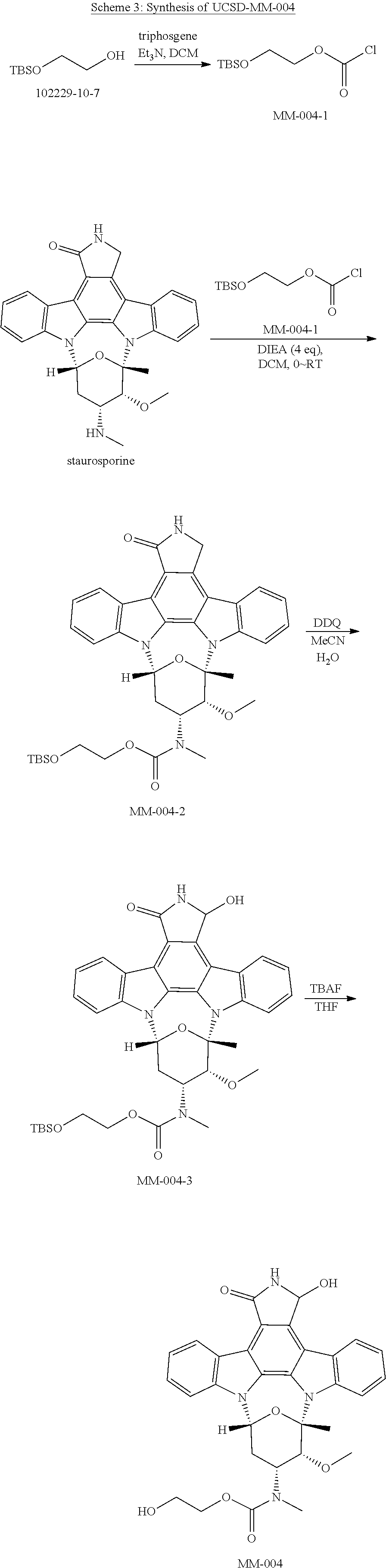

### Experimental Procedures:

### Synthesis of MM-002

### Procedure for Preparation of MM-002-1

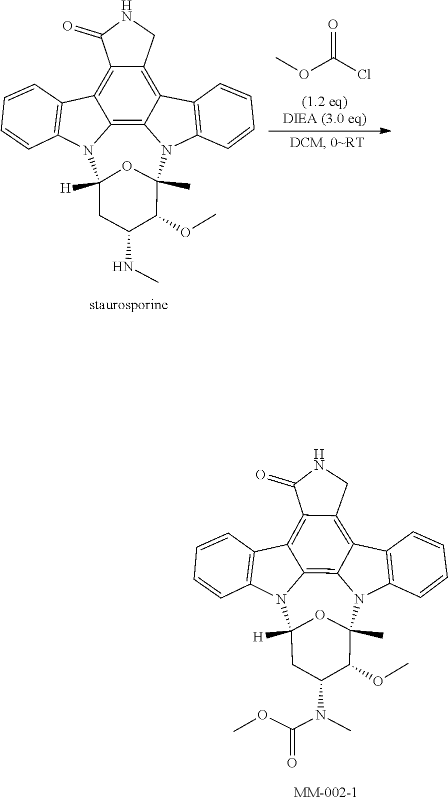

To a solution of staurosporine (100 mg, 0.21 mmol) in DCM (10 mL) at 0° C., was added N,N-diisopropylethylamine (83 mg, 0.65 mmol). After stirring for 5 min, a solution of methyl chloroformate (24 mg, 0.26 mmol) in DCM (1 mL) was added via syringe slowly. After the addition was complete, the reaction was stirred at RT for 2 h and then concentrated to dryness. The crude residue was purified by silica gel column chromatography (100:1 DCM/MeOH) to afford MM-002-1 (110 mg, 98%) as white powder solid. TLC: Rf=0.3 (silica gel, 20:1 DCM/MeOH). ESI-MS (EI+, m/z): 525.3. ¹H NMR (400 MHz, Chloroform-d) δ 9.46 (d, J=8.0 Hz, 1H), 7.96 (d, J=7.7 Hz, 1H), 7.75 (d, J=8.5 Hz, 1H), 7.54-7.44 (m, 2H), 7.38 (dt, J=11.9, 7.5 Hz, 2H), 7.29 (d, J=8.1 Hz, 1H), 6.81-6.75 (m, 1H), 6.21 (s, 1H), 5.05 (s, 2H), 4.87 (s, 1H), 4.04 (s, 1H), 3.79 (s, 2H), 3.04 (s, 1H), 2.78 (s, 3H), 2.63 (d, J=8.3 Hz, 2H), 2.55 (s, 3H), 2.48 (s, 3H).

### Procedure for Preparation of MM-002

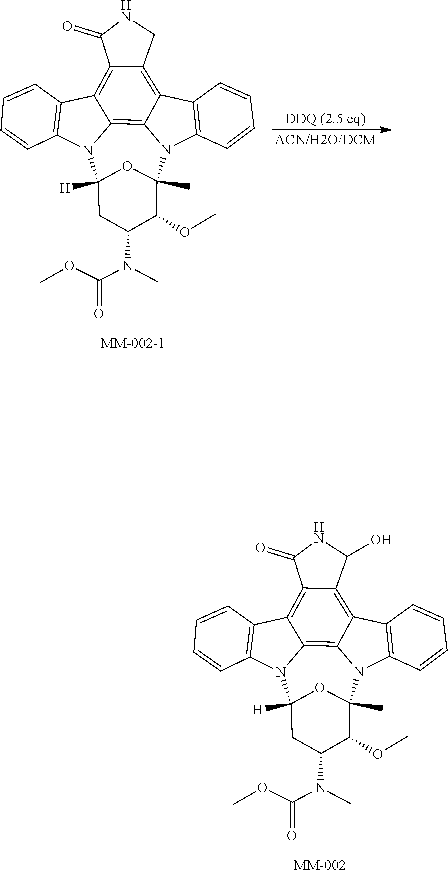

To a solution of MM-002-1 (60 mg, 0.114 mmol) in DCM (5 mL) was added a solution of DDQ (65 mg, 0.286 mmol) in acetonitrile (0.5 mL) and water (0.5 mL). The reaction mixture was stirred at RT for 4 h in the dark, before being diluted with DCM (10 mL). The mixture was washed sequentially with water (4×10 mL) and brine (5 mL), then dried over Na₂SO₄ and concentrated under reduced pressure. The crude residue was purified by prep-TLC (20:1 DCM/MeOH) to give MM-002 (17 mg, 28%) as a white powder solid. TLC: Rf=0.25 (silica gel, 20:1 DCM:MeOH). ESI-MS (EI+, m/z): 541.10. ¹H NMR (400 MHz, DMSO-d₆) δ 9.23 (d, J=7.9 Hz, 1H), 8.83 (s, 1H), 8.45 (d, J=7.8 Hz, 1H), 7.98 (d, J=8.5 Hz, 1H), 7.64 (d, J=8.2 Hz, 1H), 7.49 (td, J=8.5, 4.3 Hz, 2H), 7.32 (dt, J=9.9, 7.5 Hz, 2H), 7.01 (t, J=7.4 Hz, 1H), 6.51-6.40 (m, 2H), 4.66 (s, 1H), 4.30 (s, 1H), 3.73 (s, 3H), 2.78-2.72 (m, 1H), 2.71 (d, J=4.9 Hz, 6H), 2.35 (s, 3H), 2.24 (td, J=12.9, 6.3 Hz, 1H).

### Procedure for Preparation of MM-003-1

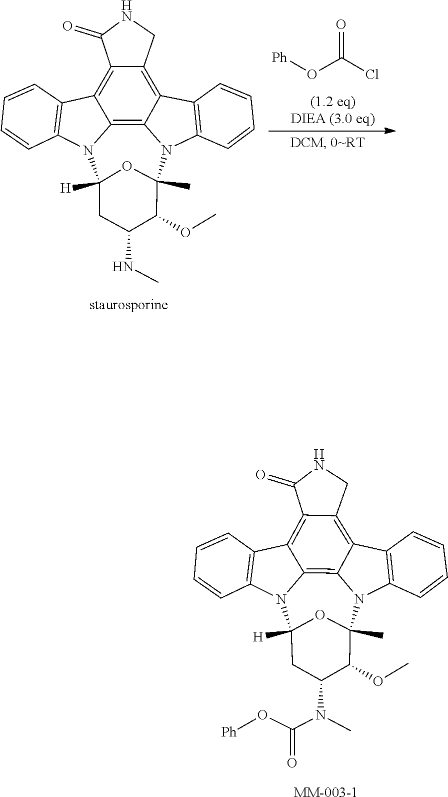

To a solution of staurosporine (100 mg, 0.21 mmol) in DCM (10 mL) at 0° C., was added N,N-diisopropylethylamine (83 mg, 0.65 mmol). After stirring for 5 min, a solution of methyl chloroformate (35 mg, 0.23 mmol) in DCM (1 mL) was added via syringe slowly. After the addition was complete, the reaction was stirred at RT for 2 h and concentrated to dryness. The crude residue was purified by silica gel column chromatography (100:1 DCM/MeOH) to afford MM-003-1 (108 mg, 88%) as white powder solid. TLC: Rf=0.5 (silica gel, 20:1 DCM/MeOH). ESI-MS (EI+, m/z): 586.2. ¹H NMR (400 MHz, Chloroform-d) δ 9.47 (d, J=8.0 Hz, 1H), 7.96 (d, J=7.7 Hz, 1H), 7.75 (d, J=8.5 Hz, 1H), 7.43 (dddd, J=42.1, 34.4, 17.2, 7.9 Hz, 8H), 7.15 (d, J=7.9 Hz, 1H), 6.81 (s, 1H), 6.31 (s, 1H), 5.05 (s, 2H), 4.90 (d, J=13.8 Hz, 1H), 4.15 (s, 1H), 2.95 (d, J=33.3 Hz, 3H), 2.74 (d, J=17.3 Hz, 2H), 2.62 (d, J=31.6 Hz, 3H), 2.47 (s, 3H).

### Procedure for Preparation of MM-003

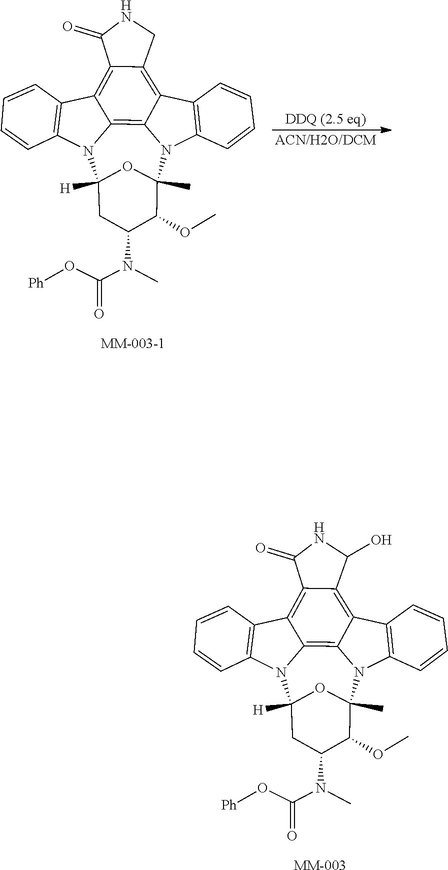

To a solution of MM-003-1 (108 mg, 0.19 mmol) in DCM (5 mL) was added a solution of DDQ (106 mg, 0.47 mmol) in acetonitrile (0.5 mL) and water (0.5 mL). The reaction mixture was stirred at RT for 4 h in the dark, before being diluted with DCM (10 mL). The mixture was washed sequentially with water (4×10 mL) and brine (5 mL), then dried over Na₂SO₄ and concentrated under reduced pressure. The crude residue was purified prep-TLC (20:1 DCM/MeOH) to give MM-003 (30 mg, 22%) as a white powder solid. TLC: Rf=0.35 (silica gel, 20:1 DCM:MeOH). ESI-MS (EI+, m/z): 603.20. ¹H NMR (400 MHz, DMSO-d₆) δ 9.23 (s, 1H), 8.84 (s, 1H), 8.47 (s, 1H), 8.01 (s, 1H), 7.67 (s, 1H), 7.50 (s, 4H), 7.32 (s, 4H), 7.19 (s, 1H), 7.05 (s, 1H), 6.48 (s, 2H), 4.76 (d, J=11.3 Hz, 1H), 4.46 (d, J=35.5 Hz, 1H), 2.88 (d, J=29.8 Hz, 6H), 2.80 (s, 1H), 2.35 (s, 4H).

### Procedure for Preparation of MM-004-1

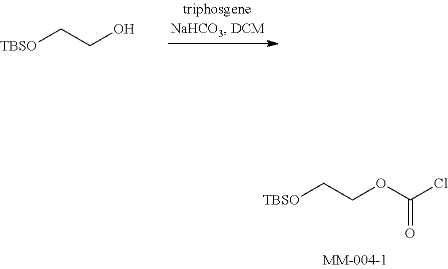 [SMILES: CC(C)(C)[Si](C)(C)OCCO ; CC(C)(C)[Si](C)(C)OCCOC(=O)Cl]

To a solution of triphosgene (500 mg, 1.68 mmol) and DMAP (2 mg) in DCM (10 mL) at 0° C., was added a solution of 2-((tert-butyldimethylsilyl)oxy)ethan-1-ol (714 mg, 4.20 mmol) in DCM (2 mL). The reaction mixture was stirred at 0° C. for 1 h and then warmed to RT and stirred for a further 2 h. The obtained chloroformate solution (˜0.35 M in DCM) was used directly in the next step.

### Procedure for Preparation of MM-004-2

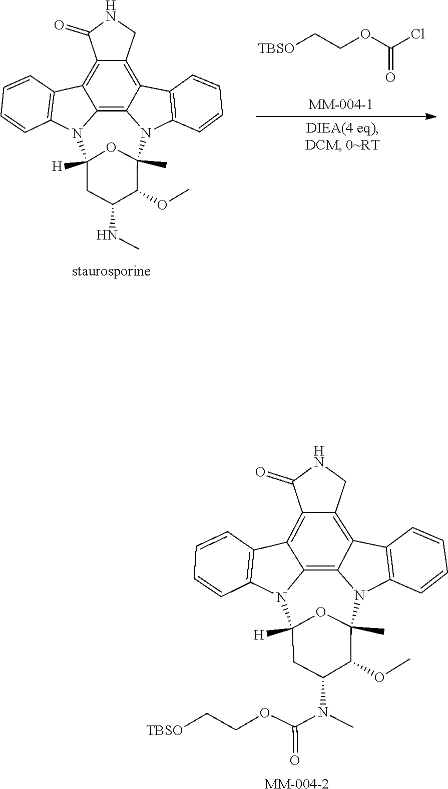

To a solution of staurosporine (200 mg, 0.42 mmol) in DCM (10 mL) at 0° C., was added N,N-diisopropylethylarnine (166 mg, 1.30 mmol). After stirring for 5 min, a solution of MM-004-1 (0.35 M in DCM, 1.44 mL, 0.50 mmol) was added via syringe slowly. After the addition was complete, the reaction was stirred at RT for 2 h (TLC indicated almost 50% SM remained). Another portion of MM-004-1 (0.35 M in DCM, 1.44 mL, 0.50 mmol) was added via syringe slowly and the mixture was stirred at RT for further 2 h, then concentrated to dryness. The crude residue was purified by silica gel column chromatography (3:1 to 1:1 PE/EtOAc) to afford MM-004-2 (200 mg, 88%) as white powder solid. TLC: Rf=0.5 (silica gel, 2:1 PE/EtOAc). ESI-MS (EI+, m/z): 669.2. ¹H NMR (400 MHz, DMSO-d₆) δ 9.29 (d, J=8.0 Hz, 1H), 8.59 (s, 1H), 8.06 (d, J=7.8 Hz, 1H), 7.99 (s, 1H), 7.64 (d, J=8.2 Hz, 1H), 7.49 (t, J=7.6 Hz, 2H), 7.36 (t, J=7.5 Hz, 1H), 7.30 (t, J=7.5 Hz, 1H), 7.01 (s, 1H), 5.00 (s, 2H), 4.64 (d, J=19.7 Hz, 1H), 4.27 (s, 1H), 4.20-4.10 (m, 2H), 3.99-3.86 (m, 2H), 2.75-2.68 (m, 7H), 2.36 (s, 3H), 2.25 (s, 1H), 0.86 (d, J=8.2 Hz, 9H), 0.04 (d, J=2.6 Hz, 6H).

### Procedure for Preparation of MM-004-3

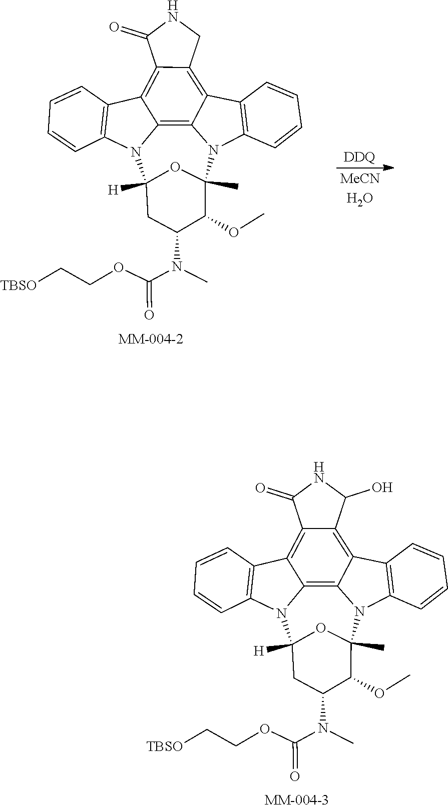

To a solution of MM-004-2 (150 mg, 0.22 mmol) in DCM (5 mL) was added a solution of DDQ (127 mg, 0.56 mmol) in acetonitrile (0.5 mL) and water (0.5 mL). The reaction mixture was stirred at RT for 4 h in the dark, before being diluted with DCM (10 mL). The mixture was washed sequentially with water (4×10 mL) and brine (5 mL), then dried over Na₂SO₄ and concentrated under reduced pressure. The crude residue was purified prep-TLC (1:1 PE/EtOAc) to afford MM-004-3 (30 mg, 22%) as a waxy solid. TLC: Rf=0.48 (silica gel, 2:1 PE:EtOAc). ESI-MS (EI+, m/z): 685.20.

### Procedure for Preparation of MM-004

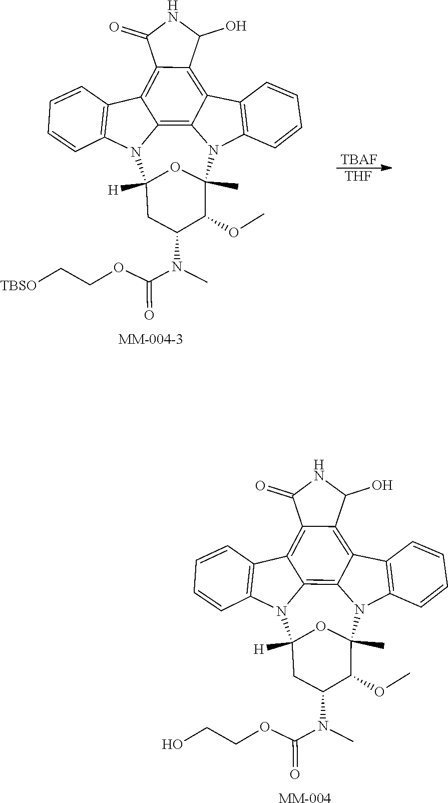

To a solution of MM-004-3 (40 mg, 0.058 mmol) in THF (2 mL) was added a solution of TBAF (0.10 mL, 0.10 mmol, 1M) in THF. The reaction mixture was stirred at RT for 2 h and then diluted with EtOAc (10 mL). The organic fraction was washed sequentially with water (4×10 mL) and brine (5 mL), then dried over Na₂SO₄ and concentrated to dryness. The crude residue was purified prep-TLC (20:1 DCM:MeOH) to give MM-004 (8 mg, 22%) as a white solid. TLC: Rf=0.25 (silica gel, 20:1 DCM:MeOH). ESI-MS (EI+, m/z): 571.20. ¹H NMR (400 MHz, DMSO-d₆) δ 9.23 (d, J=7.9 Hz, 1H), 8.83 (s, 1H), 8.47 (dd, J=18.9, 7.9 Hz, 1H), 7.96 (s, 1H), 7.65 (t, J=9.1 Hz, 1H), 7.49 (q, J=7.9 Hz, 2H), 7.32 (q, J=8.5, 7.8 Hz, 2H), 7.01 (s, 1H), 6.46 (d, J=3.5 Hz, 2H), 4.88 (d, J=62.2 Hz, 1H), 4.66 (s, 1H), 4.33 (s, 1H), 4.12 (s, 2H), 3.67 (d, J=51.1 Hz, 2H), 2.75 (d, J=26.3 Hz, 7H), 2.35 (d, J=7.1 Hz, 3H), 2.24 (s, 1H).

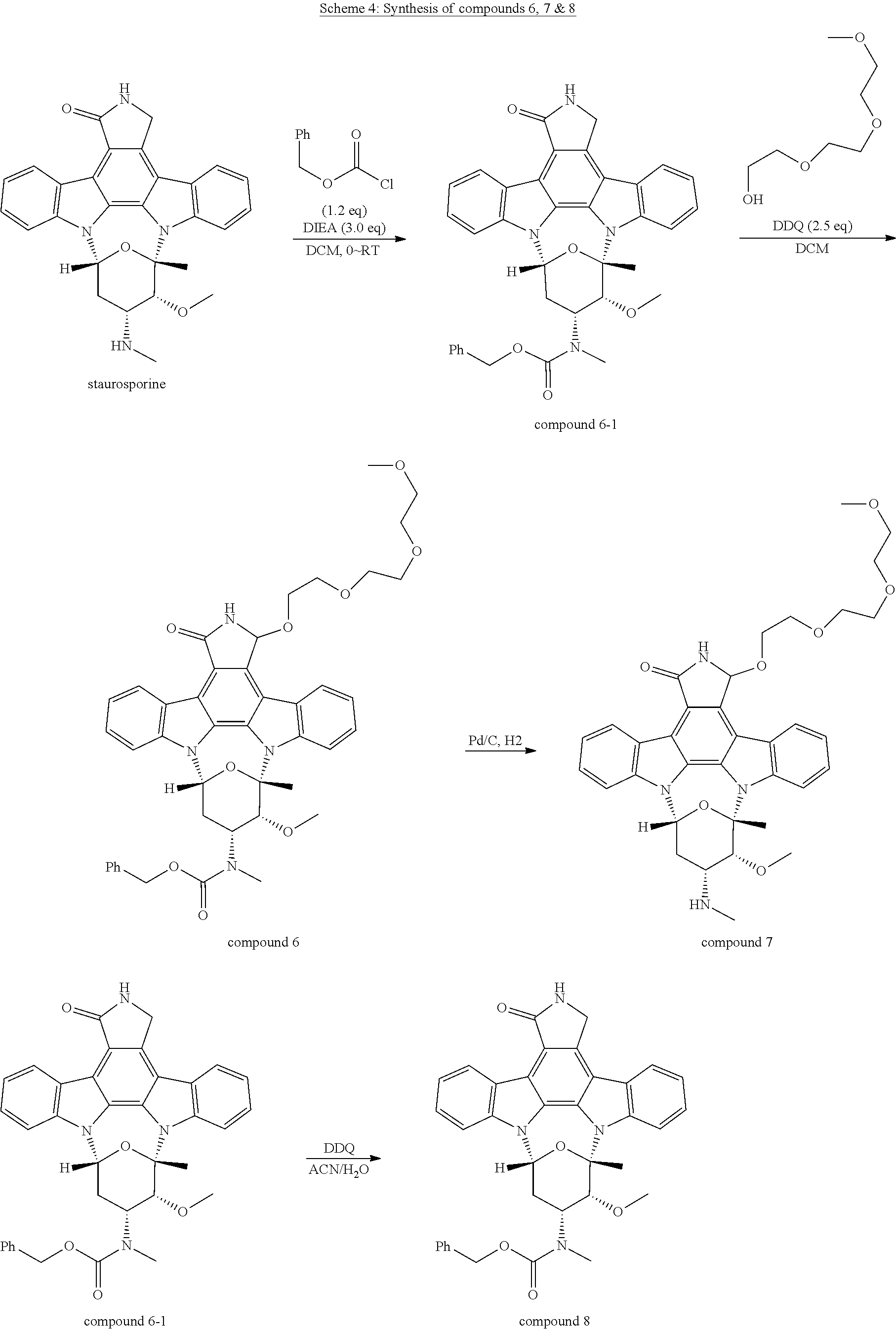

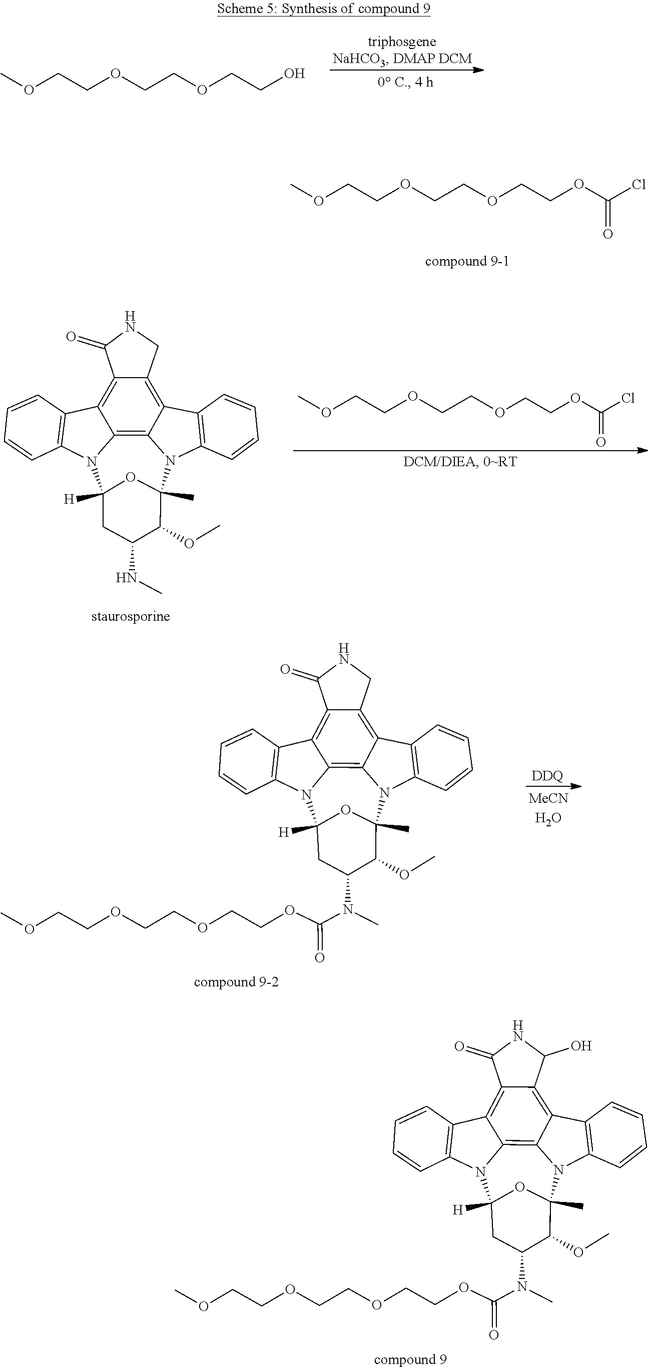

### Experimental Procedures:

### Procedure for Preparation of Compound 6-1

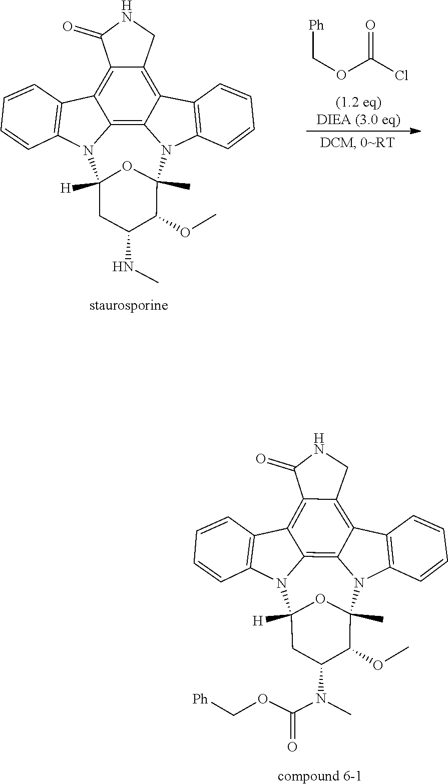

To a solution of staurosporine (200 mg, 0.43 mmol) in DCM (10 mL) at 0° C., was added N,N-diisopropylethylamine (83 mg, 0.65 mmol). After stirring for 5 min, a solution of benzyl chloroformate (110 mg, 0.64 mmol) in DCM (1 mL) was added via syringe slowly. After the addition was complete, the reaction was stirred at RT for 2 h and then concentrated to dryness. The crude residue was diluted with H₂O (10 mL) and Et₂O (10 mL) and organic fraction was filtered and concentrated to dryness to afford compound 6-1 (260 mg, 98%) as a pale-yellow solid. TLC: Rf=0.3 (20:1 DCM/MeOH). LCMS (ESI): m/z 601.1 [M+H]¹⁺. ¹H NMR (400 MHz, CDCl₃) δ 9.45 (d, J=8.0 Hz, 1H), 7.93 (d, J=7.6 Hz, 1H), 7.75 (d, J=8.4 Hz, 1H), 7.53-7.33 (m, 11H), 6.74 (s, 1H), 6.33 (s, 1H), 5.26-5.13 (m, 2H), 5.03 (s, 2H), 4.86 (d, J=13.8 Hz, 1H), 4.06 (s, 1H), 3.63 (s, 1H), 3.06 (s, 1H), 2.81 (s,3H), 2.65-2.58 (m, 1H), 2.51 (d, J=20.8 Hz, 6H), 2.19 (s, 1H).

### Procedure for Preparation of Compound 8

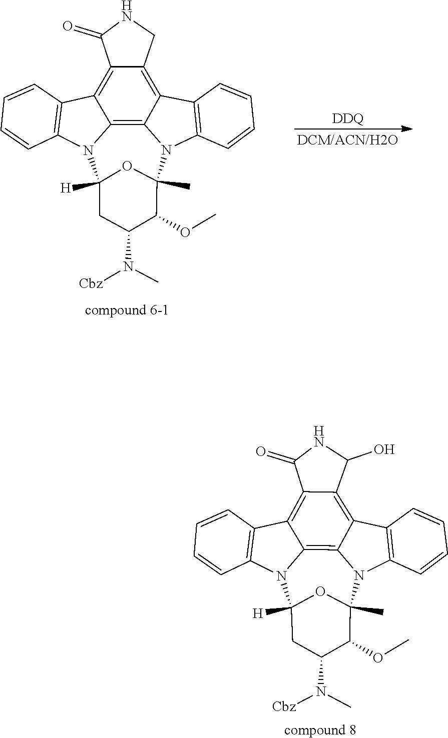

To a solution of compound 6-1 (60 mg, 0.10 mmol) in DCM (5 mL), was added a solution of DDQ (57 mg, 0.25 mmol) in 1:1 acetonitrile/H₂O (1 mL). The reaction mixture was stirred at RT for 4 h in the dark and then diluted with DCM (10 mL). The organic fraction was washed sequentially with 5% aq. Na₂S₂O₃ (2×10 mL) and brine (10 mL), then dried over Na₂SO₄ and concentrated under reduced pressure. The crude residue was purified by prep-TLC (20:1 DCM/MeOH) to afford compound 8 (17 mg, 27%) as a white powder solid. TLC: Rf=0.26 (20:1 DCM:MeOH). LCMS (ESI): m/z 617.3 [M+H]¹⁺. ¹H NMR (400 MHz, DMSO-d₆) δ 9.22 (d, J=7.9 Hz, 1H), 8.83 (s, 1H), 8.44 (d, J=7.9 Hz, 1H), 7.96 (s, 1H), 7.63-7.25 (m, 10H), 7.00 (s, 1H), 6.50-6.39 (m, 2H), 5.20 (s, 2H), 4.68 (s, 1H), 4.30 (s, 1H), 2.74 (s, 7H), 2.39-2.14 (m, 4H).

### Procedure for Preparation of Compound 6

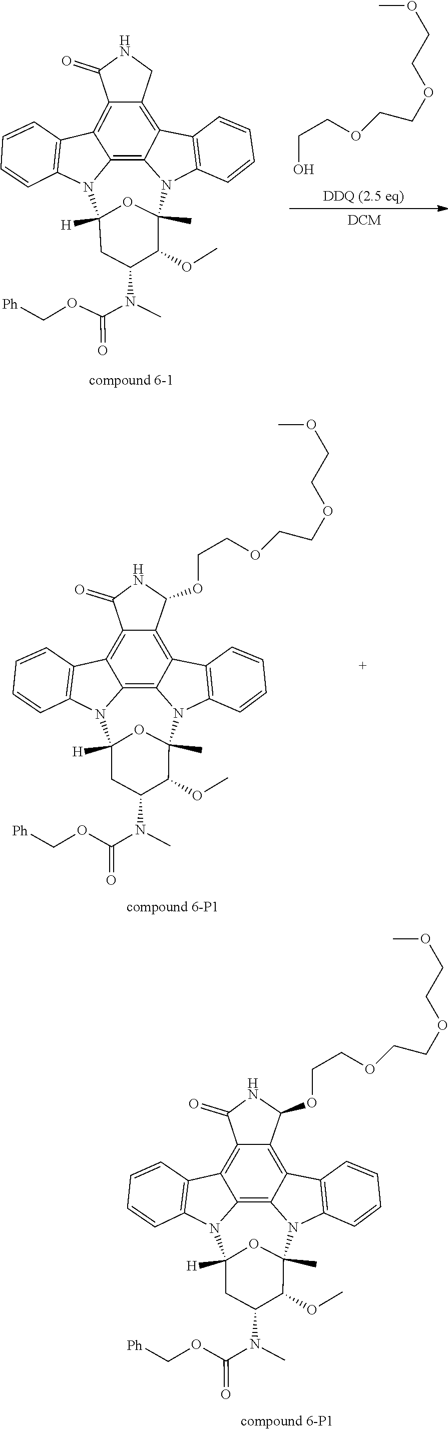

To a solution of compound 6-1 (200 mg, 0.33 mmol) and triethylene glycol monomethyl ether (500 mg, 3.05 mmol) in DCM (5 mL) at 0° C., was added and a solution of DDQ (106 mg, 0.47 mmol) in acetonitrile (0.5 mL) slowly via syringe. The reaction mixture was stirred at RT for 4 h in the dark and then diluted with DCM (30 mL). The organic fraction was washed sequentially with 2% aq. Na₂S₂O₃ (2×20 mL) and brine (20 mL), then dried over Na₂SO₄ and concentrated to dryness. The crude residue was purified by flash column chromatography (20:1 DCM/MeOH) and then prep-TLC (20:1 DCM/MeOH) to afford the epimers compound 6-P1 (22 mg, 9%) and compound 6-P2 (20 mg, 9%) as white solids. TLC: Rf=0.45 (20:1 DCM/MeOH).

### QC Data of Compound 6-P1:

LCMS (ESI): m/z 763.5 [M+H]¹⁺. ¹H NMR (400 MHz, DMSO-d₆) δ 9.20 (d, J=8.0 Hz, 1H), 9.09-9.01 (m, 1H), 8.40-8.32 (m, 1H), 7.93 (d, J=34.1 Hz, 1H), 7.64 (d, J=8.1 Hz, 1H), 7.57-7.26 (m, 9H), 7.00 (s, 1H), 6.54 (d, J=1.5 Hz, 1H), 5.18 (d, J=11.5 Hz, 2H), 4.64 (d, J=34.4 Hz, 1H), 4.29 (s, 1H), 3.84-3.73 (m, 1H), 3.54 (d, J=9.3 Hz, 3H), 3.49-3.41 (m, 7H), 3.35 (t, J=3.2 Hz, 2H), 3.17 (s, 3H), 2.70 (d, J=25.5 Hz, 7H), 2.41-2.15 (m, 4H).

### QC Data of Compound 6-P2:

LCMS (ESI): m/z 763.5 [M+H]¹⁺. ¹H NMR (400 MHz, Dmso-d₆) δ 9.20 (d, J=7.9 Hz, 1H), 9.11-9.03 (m, 1H), 8.39 (d, J=7.9 Hz, 1H), 7.95 (s, 1H), 7.68-7.27 (m, 10H), 7.00 (s, 1H), 6.54 (d, J=1.6 Hz, 1H), 5.19 (s, 2H), 4.64 (d, J=34.9 Hz, 1H), 4.27 (s, 1H), 3.82-3.72 (m, 1H), 3.57-3.47 (m, 3H), 3.46-3.39 (m, 7H), 3.35 (d, J=4.2 Hz, 3H), 3.17 (s, 3H), 2.72 (d, J=17.9 Hz, 7H), 2.41-2.10 (m, 4H).

### Procedure for Preparation of Compound 7

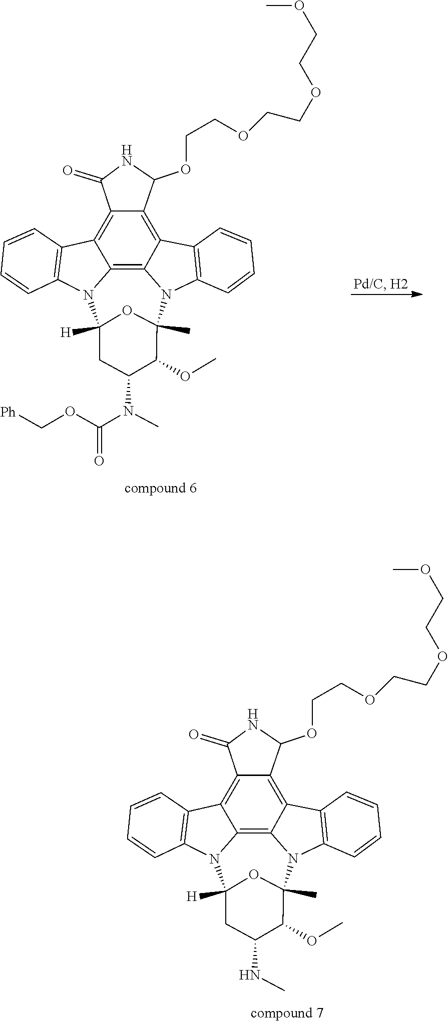 [SMILES: COCCOCCOCCOC1NC(=O)c2c1c1c3ccccc3n3c1c1c2c2ccccc2n1[C@H]1C[C@@H](N(C)C(=O)OCc2ccccc2)[C@@H](OC)[C@]3(C)O1 ; "Pd/C, H2" ; CN[C@@H]1C[C@H]2O[C@@](C)([C@@H]1OC)n1c3ccccc3c3c4c(c5c6ccccc6n2c5c31)C(=O)NC4OCCOCCOCCOC]

To a solution of compound 6 (racemic 130 mg, 0.17 mmol) in 1:3 DCM/MeOH (20 mL) was added 10% wt. Pd/C (18 mg, 0.17 mmol). The reaction mixture was degassed with H₂ and then stirred at RT for 24 h under an atmosphere of H₂. The catalyst was removed by filtration and the filtrate concentrated under reduced pressure. The crude residue was purified by prep-TLC (20:1 DCM/MeOH) to afford compound 7 (80 mg, 75%) as a white solid. TLC: Rf=0.25 (20:1 DCM/MeOH). LCMS (ESI): m/z 629.3 [M+H]¹⁺. ¹H NMR (400 MHz, DMSO-d₆) δ 9.20 (d, J=7.9 Hz, 1H), 8.97 (s, 1H), 8.27 (d, J=7.6 Hz, 1H), 7.97 (d, J=8.6 Hz, 1H), 7.61 (d, J=8.2 Hz, 1H), 7.48 (t, J=7.6 Hz, 1H), 7.41 (t, J=7.9 Hz, 1H), 7.27 (dt, J=12.2, 7.5 Hz, 2H), 6.71 (d, J=3.7 Hz, 1H), 6.48 (d, J=1.5 Hz, 1H), 4.08 (d, J=3.6 Hz, 1H), 3.77 (d, J=8.8 Hz, 1H), 3.54 (d, J=2.7 Hz, 3H), 3.49-3.41 (m, J=5.6, 5.0 Hz, 6H), 3.40-3.33 (m, 5H), 3.28 (s, 1H), 3.19 (d, J=5.1 Hz, 3H), 2.56-2.46 (m, 2H), 2.31 (s, 3H), 1.46 (d, J=8.0 Hz, 3H).

### Procedure for Preparation of Compound 9-1

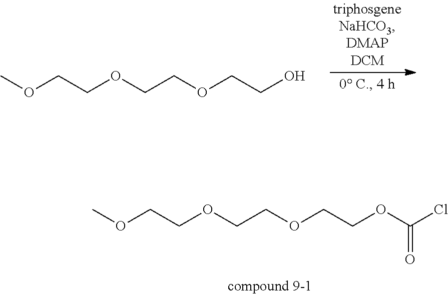

To a solution of triphosgene (317 mg, 1.07 mmol) and DMAP (2 mg) in DCM (10 mL) at 0° C., was added NaHCO₃ (1.54 g, 18.30 mmol). The mixture was stirred for 10 min and then a solution of triethylene glycol monomethyl ether (500 mg, 3.05 mmol) in DCM (2 mL) was added slowly via syringe. The reaction mixture was stirred at 0° C. for 1 h and then warmed to RT and stirred for a further 2 h. The obtained solution (0.25 M in DCM) was telescoped directly into the subsequent step.

### Procedure for Preparation of Compound 9-2

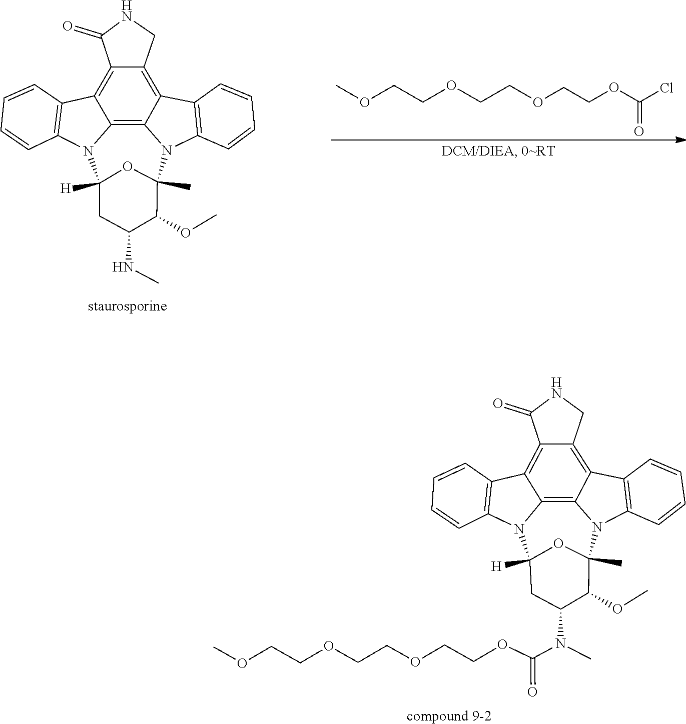 [SMILES: CN[C@@H]1C[C@H]2O[C@@](C)([C@@H]1OC)n1c3ccccc3c3c4c(c5c6ccccc6n2c5c31)C(=O)NC4 ; COCCOCCOCCOC(=O)Cl ; COCCOCCOCCOC(=O)N(C)[C@@H]1C[C@H]2O[C@@](C)([C@@H]1OC)n1c3ccccc3c3c4c(c5c6ccccc6n2c5c31)C(=O)NC4 ; "compound 9-2"]

To a 0° C. solution of staurosporine (100 mg, 0.21 mmol) in DCM (10 mL) was added N,N-diisopropylethylamine (109 mg, 0.84 mmol). After stirring for 5 min, a solution of compound 9-1 (0.25 M in DCM, 2.52 mL, 0.63 mmol) was added via syringe slowly. After the addition, the reaction was stirred at RT for 2 hours. The reaction mixture was concentrated to dryness. The residue was purified by silica gel column chromatography (Petro Ether/EtOAc=3/1 to 1/1, v/v) to afford compound 9-2 (120 mg, 85%) as pale-yellow solid. TLC: Rf=0.3 (2:1 petroleum ether/EtOAc=2:1). LCMS (ESI): m/z 657.3 [M+H]¹⁺. ¹H NMR (400 MHz, DMSO-d₆) δ 9.28 (d, J=7.9 Hz, 1H), 8.59 (s, 1H), 8.06 (d, J=7.8 Hz, 1H), 7.98 (d, J=8.5 Hz, 1H), 7.63 (d, J=8.2 Hz, 1H), 7.49 (td, J=7.9, 3.1 Hz, 2H), 7.36 (t, J=7.5 Hz, 1H), 7.30 (t, J=7.5 Hz, 1H), 7.01 (t, J=7.3 Hz, 1H), 5.00 (s, 2H), 4.65 (s, 1H), 4.25 (d, J=29.1 Hz, 3H), 3.83-3.41 (m, 10H), 3.23 (d, J=6.1 Hz, 3H), 2.70 (s, 7H), 2.36 (s, 3H), 2.24 (td, J=12.9, 6.1 Hz, 1H).

### Procedure for Preparation of Compound 9

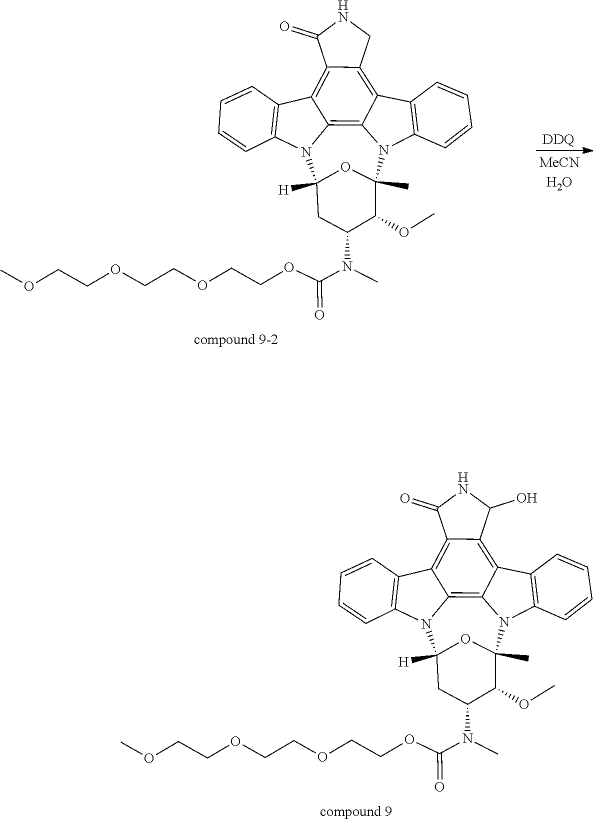

To a solution of compound 9-2 (120 mg, 0.18 mmol) in DCM (5 mL), was added a solution of DDQ (104 mg, 0.46 mmol) in 1:1 MeCN/H₂O (1 mL). The reaction mixture was stirred at RT for 4 h in the dark and then diluted with DCM (10 mL). The organic fraction was washed sequentially with H₂O (4×10 mL) and brine (5 mL), then dried over Na₂SO₄ and concentrated under reduced pressure. The crude residue was purified by prep-TLC (1:1 petroleum ether/EtOAc) to afford compound 9 (20 mg, 17%) as a white solid. TLC: Rf=0.30 (10:1 DCM/MeOH). LCMS (ESI): m/z 673.3 [M+H]¹⁺. ¹H NMR (400 MHz, DMSO-d₆) δ 9.23 (d, J=8.0 Hz, 1H), 8.83 (s, 1H), 8.45 (d, J=7.8 Hz, 1H), 7.95 (d, J=8.4 Hz, 1H), 7.64 (d, J=8.3 Hz, 1H), 7.49 (q, J=7.2 Hz, 2H), 7.32 (dt, J=11.1, 7.6 Hz, 2H), 7.01 (t, J=7.4 Hz, 1H), 6.47 (q, J=10.1 Hz, 2H), 4.64 (s, 1H), 4.26 (d, J=33.1 Hz, 3H), 3.77-3.53 (m, I0H), 3.22 (s, 3H), 2.71 (s, 7H), 2.35 (s, 3H), 2.30-2.15 (m, 1H).

### REFERENCES

Fuse et al., 2005. Review of UCN-01 development: a lesson in the importance of clinical pharmacology. J Clin Pharmacol vol. 45,4: pp. 394-03.
Hotte et al., 2006. Phase I trial of UCN-01 in combination with topotecan in patients with advanced solid cancers: a Princess Margaret Hospital Phase II Consortium study. Ann Oncol vol. 17, 2: pp. 334-40.
Mull et al., 2020. Specific, reversible G1 arrest by UCN-01 in vivo provides cytostatic protection of normal cells against cytotoxic chemotherapy in breast cancer. Br J Cancer vol. 122, 6: pp. 812-22.
Pollack et al., 1996. Blocking of glioma proliferation in vitro and in vivo and potentiating the effects of BCNU and cisplatin: UCN-01, a selective protein kinase C inhibitor. J Neurosurg vol. 84 6: pp. 1024-32.
Schneider et al, 2020. Preclinical in vivo evaluation of novel radiosensitizers by local tumor control experiments. In: Willers, Eke, eds. Molecular Targeted Radiosensitizers: Opportunities and Challenges. Cham, Switzerland: Springer Nature; 2020; pp. 137-160.
Signore et al., 2014. Combined PDK1 and CHK1 inhibition is required to kill glioblastoma stem-like cells in vitro and in vivo. Cell Death Dis vol. 5,5 e1223.
Xiao et al., 2002. 7-Hydroxystaurosporine (UCN-01) preferentially sensitizes cells with a disrupted TP53 to gamma radiation in lung cancer cell lines. Radiat Res vol. 158, 1: pp. 84-93.

### P EMBODIMENTS

P Embodiment 1. A compound, or a pharmaceutically acceptable salt thereof, having the formula:

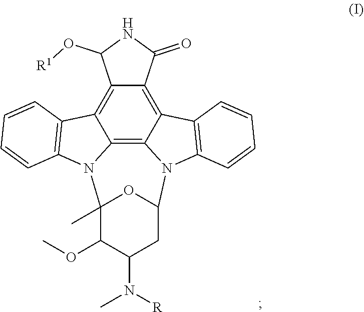

wherein
R and R¹ are independently hydrogen, halogen, —CX²₃, —CHX²₂, —CH₂X²,
—OCX²₃, —OCHX²₂, —OCH₂X², —CN, —SOₙ₂R², —SOᵥ₂NR²R³, —NR⁴NR²R³, —ONR²R³,
—NR⁴C(O)NR²R³, —N(O)ₘ₂, —NR²R³, —C(O)R², —C(NH)R², —C(O)OR², —OC(O)R 2, —OC(O)OR²,
—C(O)NR²R³, —OC(O)NR²R³, —OR², —SR², —NR⁴SO₂R², —NR⁴C(O)R², —NR⁴C(O)OR²,
—NR²OR³, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R², R³, and R⁴ are independently hydrogen, halogen, —CCl₃, —CBr₃, —CF₃, —CI₃,
—CHCl₂, —CHBr₂, —CHF₂, —CHI₂, —CH₂Cl, —CH₂Br, —CH₂F, —CH₂I, —CN, —OH, —NH₂, —COOH,
—CONH₂, —OCCl₃, —OCF₃, —OCBr₃, —OCI₃, —OCHCl₂, —OCHBr₂, —OCHI₂, —OCH F₂, —OCH₂Cl,
—OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R³ substituents bonded to the same nitrogen atom may optionally be joined to form a substituted or unsubstituted heterocycloalkyl or substituted or unsubstituted heteroaryl;
m2 and v2 are independently 1 or 2;
n2 is an integer from 0 to 4; and
each X² is independently —Cl, —Br, —I, or —F;
wherein R and R¹ are not both hydrogen.
P Embodiment 2. The compound of embodiment 1, wherein R is hydrogen,

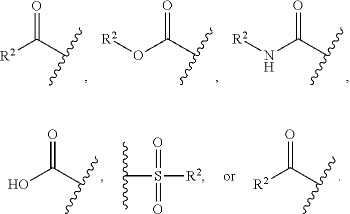

P Embodiment 3. The compound of embodiment 2, wherein R² is hydrogen, substituted or unsubstituted C₁-C₄ alkyl, substituted or unsubstituted 3 to 6 membered heterocycloalkyl, unsubstituted phenyl, unsubstituted 5 to 6 membered heteroaryl, or —(CH₂CH₂O)ₙ-(unsubstituted C₁-C₄ alkyl), wherein n is an integer from 1 to 12.
P Embodiment 4. The compound of embodiment 2, wherein R² is hydrogen, unsubstituted methyl, —CH₂CH₂OH, —CH₂-(unsubstituted phenyl), unsubstituted piperidinyl, substituted dioxanyl, unsubstituted phenyl, unsubstituted imidazolyl, unsubstituted oxazolyl, unsubstituted thiazolyl, unsubstituted pyrazinyl, —(CH₂CH₂O)₂—CH₃, —(CH₂CH₂O)₃—CH₃, or —(CH₂CH₂O)₄—CH₃.
P Embodiment 5. The compound of embodiment 1, wherein R is hydrogen,

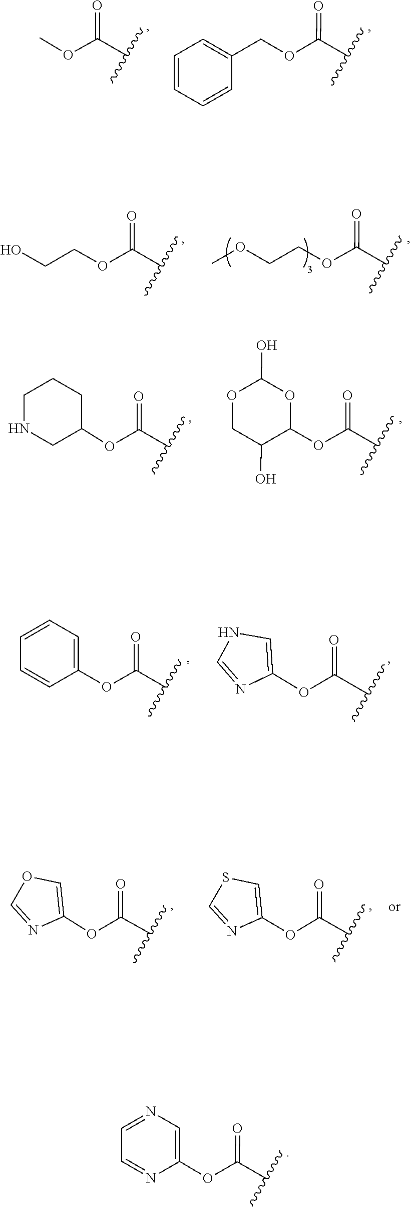

P Embodiment 6. The compound of any one of embodiments 1 to 5, wherein R¹ is hydrogen, unsubstituted C₁-C₄ alkyl, unsubstituted phenyl, or —(CH₂CH₂O)ₙ-(unsubstituted C₁-C₄ alkyl), wherein n is an integer from 1 to 12.
P Embodiment 7. The compound of any one of embodiments 1 to 5, wherein R¹ is hydrogen, unsubstituted methyl, unsubstituted butyl, unsubstituted phenyl, or —(CH₂CH₂O)₃—CH₃.
P Embodiment 8. The compound of embodiment 1, wherein R or R¹ is a cleavable moiety.
P Embodiment 9. The compound of embodiment 1, wherein R and R¹ are each a cleavable moiety.
P Embodiment 10. The compound of embodiment 8 or 9, wherein the cleavable moiety is

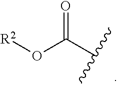

P Embodiment 11. The compound of embodiment 10, wherein R² is unsubstituted C₁-C₄ alkyl, unsubstituted phenyl, or —(CH₂CH₂O)ₙ-(unsubstituted C₁-C₄ alkyl), wherein n is an integer from 1 to 12.
P Embodiment 12. The compound of embodiment 10, wherein R² is unsubstituted methyl, unsubstituted butyl, unsubstituted phenyl, —(CH₂CH₂O)₂—CH₃, —(CH₂CH₂O)₃—CH₃, —(CH₂CH₂O)₄—CH₃, or —(CH₂CH₂O)₃—CH₂CH₃.
P Embodiment 13. The compound of embodiment 1, having the formula:

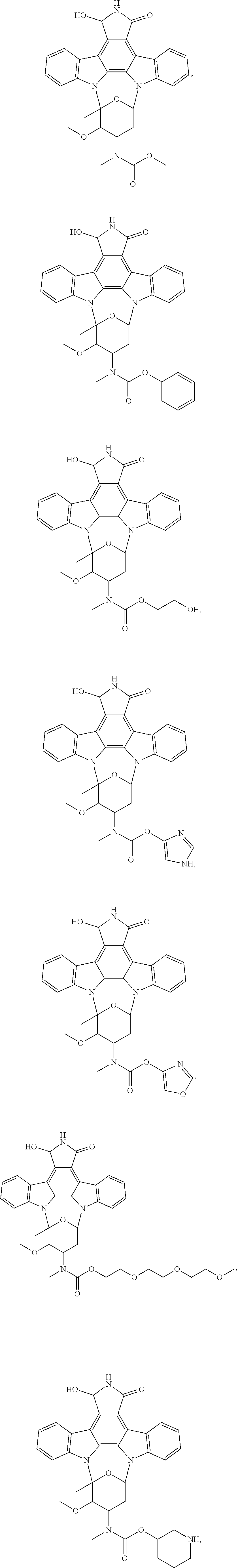

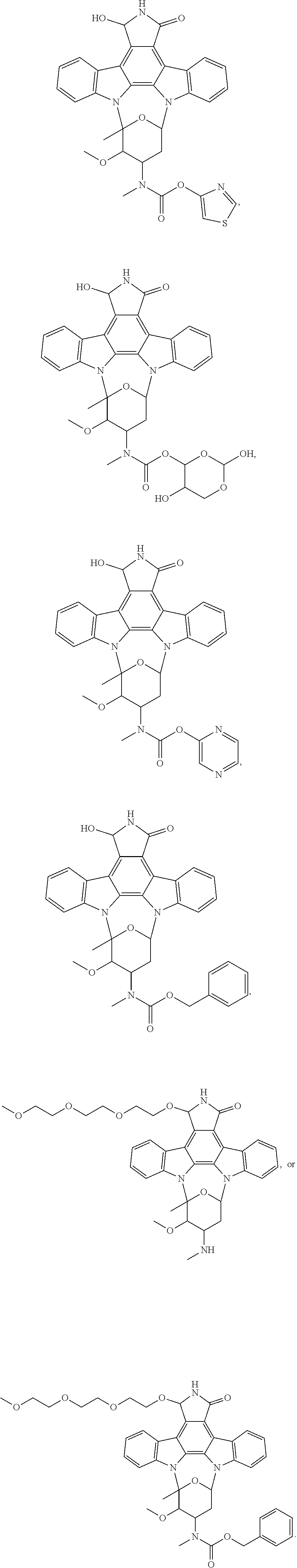 [SMILES: COC1C(N(C)C(=O)Oc2cscn2)CC2OC1(C)n1c3ccccc3c3c4c(c5c6ccccc6n2c5c31)C(=O)NC4O ; COC1C(N(C)C(=O)OC2OC(O)OCC2O)CC2OC1(C)n1c3ccccc3c3c4c(c5c6ccccc6n2c5c31)C(=O)NC4O ; COC1C(N(C)C(=O)Oc2cnccn2)CC2OC1(C)n1c3ccccc3c3c4c(c5c6ccccc6n2c5c31)C(=O)NC4O ; COC1C(N(C)C(=O)OCc2ccccc2)CC2OC1(C)n1c3ccccc3c3c4c(c5c6ccccc6n2c5c31)C(=O)NC4O ; CNC1CC2OC(C)(C1OC)n1c3ccccc3c3c4c(c5c6ccccc6n2c5c31)C(=O)NC4OCCOCCOCCOC ; COCCOCCOCCOC1NC(=O)c2c1c1c3ccccc3n3c1c1c2c2ccccc2n1C1CC(N(C)C(=O)OCc2ccccc2)C(OC)C3(C)O1]

P Embodiment 14. A pharmaceutical composition comprising the compound of embodiment 1 and a pharmaceutically acceptable excipient.
P Embodiment 15. The pharmaceutical composition of embodiment 14, wherein the compound is comprised in a stable micelle.
P Embodiment 16. The pharmaceutical composition of embodiment 15, wherein the micelle comprises a polyethylene glycol (PEG) moiety.
P Embodiment 17. A method for treating a subject suffering from cancer or a tumor, comprising administering a therapeutically effective amount of the pharmaceutical composition according to any one of embodiments 14 to 16 to the subject.
P Embodiment 18. The method of embodiment 17, further comprising administering to the subject a chemotherapy or a radiotherapy.
P Embodiment 19. The method of embodiment 17, wherein the cancer is a head and neck cancer (HNSCC), breast cancer, or brain cancer.
P Embodiment 20. The method of embodiment 17, wherein the brain cancer GBM.
P Embodiment 21. A composition comprising a stable micelle of a staurosporine analog.
P Embodiment 22. The composition of embodiment 21, wherein said stable micelle comprises a polyethylene glycol (PEG) moiety.
P Embodiment 23. The composition of embodiment 21 or 22, wherein said stable micelle comprises a staurosporine analog of the formula:

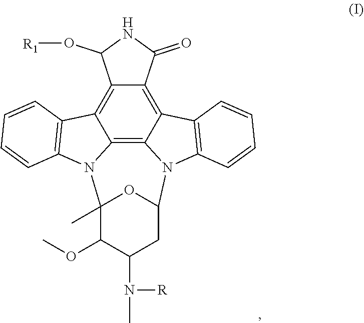

wherein R and R¹ are any chemical group.
P Embodiment 24. The composition of embodiment 23, wherein R or R¹ is

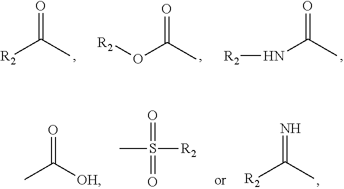

wherein R² is H, an aliphatic hydrocarbon from C₁₋₁₂, an aromatic group such as a phenyl or a heterocyclic group comprising five and six membered rings with heteroatoms O, N, S or a pegylated compound (CH₂—CH₂—O—)ₙ with n=1 to 12.
P Embodiment 25. The composition of embodiment 23, wherein said staurosporine analog is (7-hydroxystaurosporine (UCN-01) having the formula:

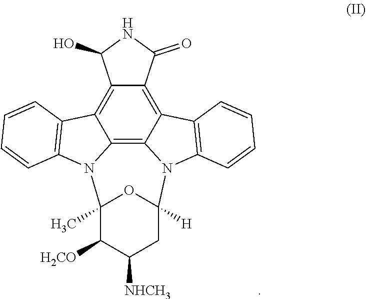

P Embodiment 26. The composition of any of embodiments 22 to 25, wherein the PEG moiety is covalently attached to R or R¹.
P Embodiment 27. The composition of any of embodiments 22 to 26, wherein the PEG moiety further comprises a linker.

What is claimed is:

1. A compound, or a pharmaceutically acceptable salt thereof, having the formula: 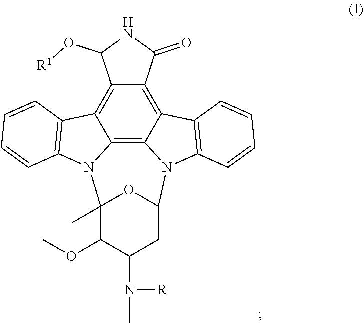
wherein
R and R¹ are independently hydrogen, halogen, —CX²₃, —CHX²₂, —CH₂X²,
—OCX²₃, —OCHX²₂, —OCH₂X², —CN, —SOₙ₂R², —SOᵥ₂NR²R³, —NR⁴NR²R³, —ONR²R³,
—NR⁴C(O)NR²R³, —N(O)ₘ₂, —NR²R³, —C(O)R², —C(NH)R², —C(O)OR², —OC(O)R 2, —OC(O)OR²,
—C(O)NR²R³, —OC(O)NR²R³, —OR², —SR², —NR⁴SO₂R², —NR⁴C(O)R², —NR⁴C(O)OR²,
—NR²OR³, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R², R³, and R⁴ are independently hydrogen, halogen, —CCl₃, —CBr₃, —CF₃, —CI₃,
—CHCl₂, —CHBr₂, —CHF₂, —CHI₂, —CH₂Cl, —CH₂Br, —CH₂F, —CH₂I, —CN, —OH, —NH₂, —COOH,
—CONH₂, —OCCl₃, —OCF₃, —OCBr₃, —OCI₃, —OCHCl₂, —OCHBr₂, —OCHI₂, —OCH F₂, —OCH₂Cl,
—OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R³ substituents bonded to the same nitrogen atom may optionally be joined to form a substituted or unsubstituted heterocycloalkyl or substituted or unsubstituted heteroaryl;
m2 and v2 are independently 1 or 2;
n2 is an integer from 0 to 4; and
each X² is independently —Cl, —Br, —I, or —F;
wherein R and R¹ are not both hydrogen.

2. The compound of claim 1, wherein R is hydrogen,

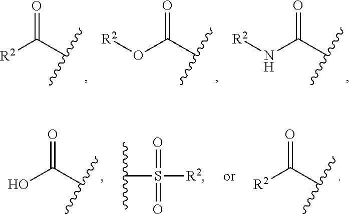

3. The compound of claim 2, wherein R² is hydrogen, substituted or unsubstituted C₁-C₄ alkyl, substituted or unsubstituted 3 to 6 membered heterocycloalkyl, unsubstituted phenyl, unsubstituted 5 to 6 membered heteroaryl, or
—(CH₂CH₂O)ₙ-(unsubstituted C₁-C₄ alkyl), wherein n is an integer from 1 to 12.

4. The compound of claim 2, wherein R² is hydrogen, unsubstituted methyl, —CH₂CH₂OH, —CH₂-(unsubstituted phenyl), unsubstituted piperidinyl, substituted dioxanyl, unsubstituted phenyl, unsubstituted imidazolyl, unsubstituted oxazolyl, unsubstituted thiazolyl, unsubstituted pyrazinyl, —(CH₂CH₂O)₂—CH₃, —(CH₂CH₂O)₃—CH₃, or
—(CH₂CH₂O)₄—CH₃.

5. The compound of claim 1, wherein R is hydrogen,

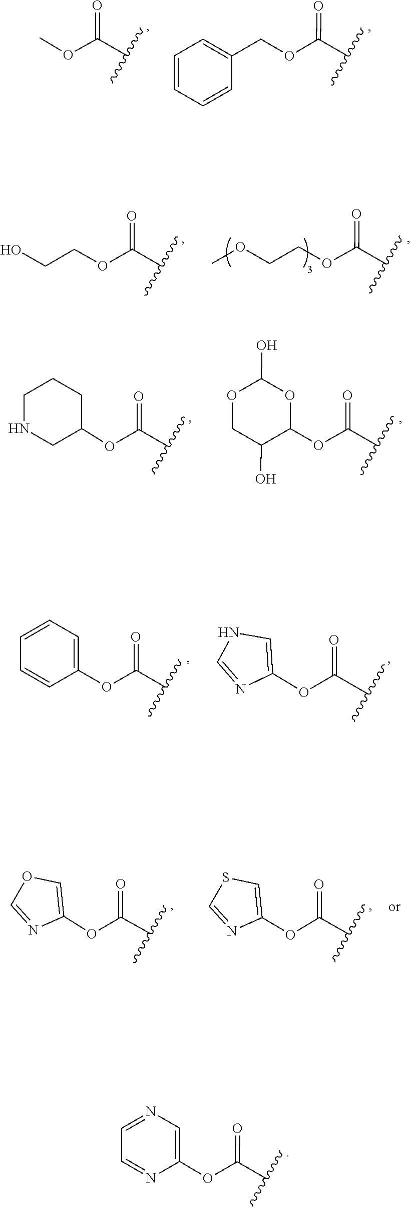

6. The compound of claim 1, wherein R¹ is hydrogen, unsubstituted C₁-C₄ alkyl, unsubstituted phenyl, or —(CH₂CH₂O)ₙ-(unsubstituted C₁-C₄ alkyl), wherein n is an integer from 1 to 12.

7. The compound of claim 1, wherein R¹ is hydrogen, unsubstituted methyl, unsubstituted butyl, unsubstituted phenyl, or —(CH₂CH₂O)₃—CH₃.

8. The compound of claim 1, wherein R or R¹ is a cleavable moiety.

9. The compound of claim 1, wherein R and R¹ are each a cleavable moiety.

10. The compound of claim 8, wherein the cleavable moiety is

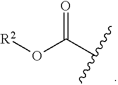

11. The compound of claim 10, wherein R² is unsubstituted C₁-C₄ alkyl, unsubstituted phenyl, or —(CH₂CH₂O)ₙ-(unsubstituted C₁-C₄ alkyl), wherein n is an integer from 1 to 12.

12. The compound of claim 10, wherein R² is unsubstituted methyl, unsubstituted butyl, unsubstituted phenyl, —(CH₂CH₂O)₂—CH₃, —(CH₂CH₂O)₃—CH₃,
—(CH₂CH₂O)₄—CH₃, or —(CH₂CH₂O)₃—CH₂CH₃.

13. The compound of claim 1, having the formula:

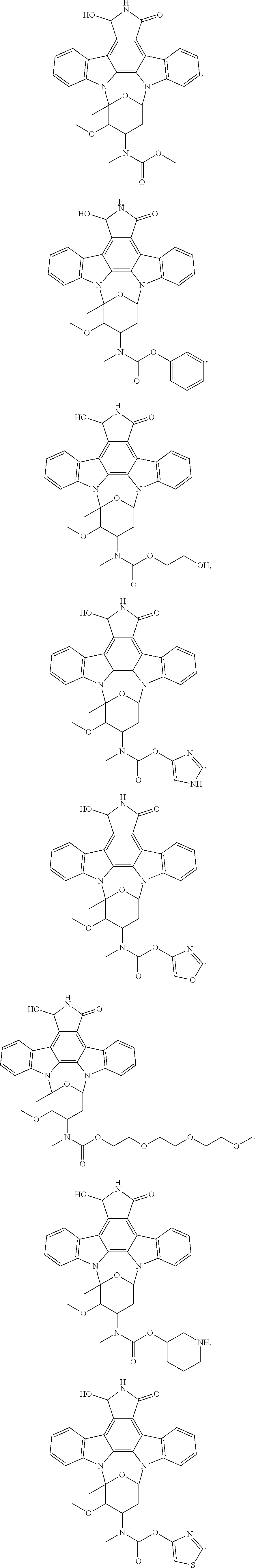

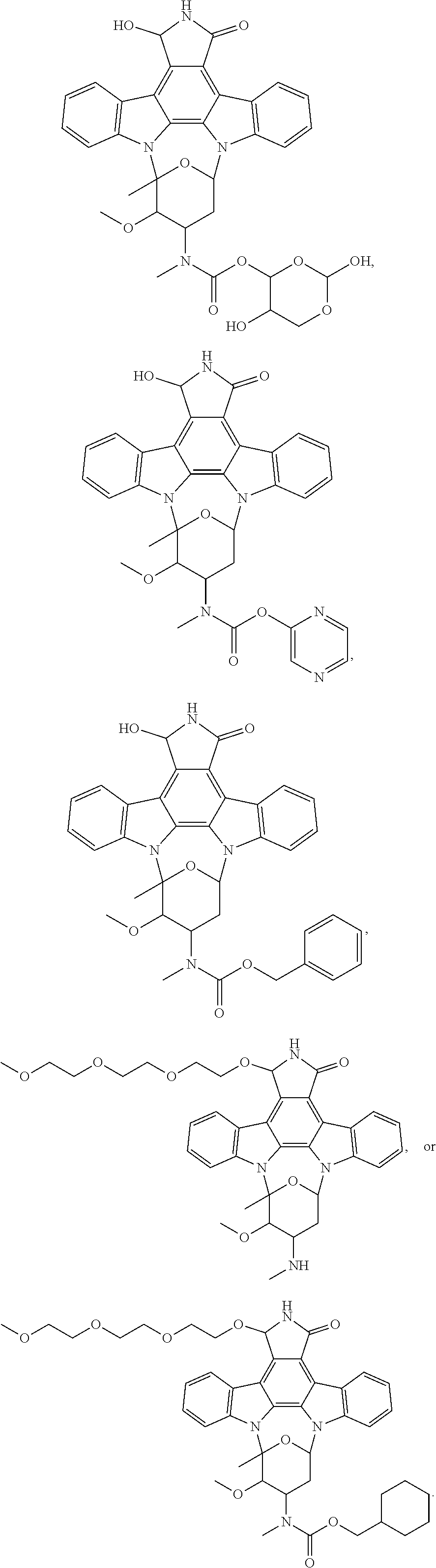

14. A pharmaceutical composition comprising the compound of claim 1 and a pharmaceutically acceptable excipient.

15. The pharmaceutical composition of claim 14, wherein the compound is comprised in a stable micelle.

16. The pharmaceutical composition of claim 15, wherein the micelle comprises a polyethylene glycol (PEG) moiety.

17. A method for treating a subject suffering from cancer or a tumor, comprising administering a therapeutically effective amount of the pharmaceutical composition according to claim 14 to the subject.

18. The method of claim 17, further comprising, administering to the subject a chemotherapy or a radiotherapy.

19. The method of claim 17, wherein the cancer is a head and neck cancer (HNSCC), breast cancer, or brain cancer.

20. The method of claim 17, wherein the brain cancer GBM.
